(19) United States
(12) Patent Application Publication
(10) Pub. No.: US 2008/0009741 A1
(43) Pub. Date: Jan. 10, 2008

(54) Ultrasonic transducer array, ultrasonic probe, ultrasonic endoscope and ultrasonic diagnostic apparatus

(75) Inventor: Hiroaki Hyuga, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Minato-ku (JP)

(21) Appl. No.: 11/802,671

(22) Filed: May 24, 2007

(30) Foreign Application Priority Data
Jun. 2, 2006 (JP) 2006-155295

(51) Int. Cl. A61B 8/14

(52) U.S. Cl. 600/459; 600/462

(57) ABSTRACT

In an ultrasonic transducer array in which plural kinds of vibrators having different shapes from one another are arranged, electric impedances of the plural kinds of element are made equal to one another. An ultrasonic transducer array, in which plural ultrasonic transducers having at least two kinds of shapes are arranged, includes a first ultrasonic transducer, and a second ultrasonic transducer having a different shape from that of the first ultrasonic transducer, and an area of an ultrasonic transmission/reception face of the second ultrasonic transducer is substantially equal to an area of an ultrasonic transmission/reception face of the first ultrasonic transducer.

### BACKGROUND OF THE INVENTION

1. Field of the invention

The present invention relates to an ultrasonic transducer array for transmission and reception of ultrasonic waves, and an ultrasonic probe and an ultrasonic endoscope including such an ultrasonic transducer array, and further, an ultrasonic diagnostic apparatus for generating ultrasonic images by using the ultrasonic probe or ultrasonic endoscope.

2. Description of a Related Art

Ultrasonic imaging technologies, which generates images showing conditions within an object to be inspected by receiving ultrasonic waves transmitted toward the object and reflected by structures (organs and so on) within the object and performing signal processing thereon, are widely used in various fields including medical fields. An apparatus for ultrasonic imaging (called an ultrasonic diagnostic apparatus, ultrasonic imaging apparatus, or the like) is provided with a probe or ultrasonic endoscope for transmission and reception of ultrasonic waves. For imaging, the probe is used in contact with the object, or the ultrasonic endoscope is inserted into the object for use.

In the ultrasonic probe and ultrasonic endoscope, vibrators (piezoelectric vibrators) each having a piezoelectric material with electrodes formed on both sides thereof are generally used as ultrasonic transducers for transmission and reception of ultrasonic waves. When an electric field is applied to the electrodes of the vibrator, the piezoelectric material expands and contracts because of piezoelectric effect and generates ultrasonic waves. Accordingly, by driving plural vibrators while delaying the timing, an ultrasonic beam focused at a desired depth can be formed. Further, the vibrator receives propagating ultrasonic waves, expands and contracts, and generates electric signals. The electric signals are used as reception signals of ultrasonic waves.

Recent years, an arrayed transducer (an ultrasonic transducer array), in which plural vibrators are arranged, has been used in the ultrasonic probe and ultrasonic endoscope. With the ultrasonic transducer array, the transmission position and direction of an ultrasonic beam can be changed by controlling the amplitudes and amounts of delay of drive signals to be respectively applied to the plural vibrators without change in the position and orientation of the probe itself. Such scan system is called a phased array system or electronic scan system.

As a related technology, Japanese Patent Application Publication JP-A-7-203592 discloses, in order to improve the beam directivity in an ultrasonic probe having high resolving power, an ultrasonic probe having plural probe segments combined and arranged in a matrix form with ultrasonic radiation faces toward a predefined direction and displacing means for respectively displacing the ultrasonic radiation faces of the respective probe segments. That is, in JP-A-7-203592, the ultrasonic transmission faces of the probe segments (vibrators) are mechanically moved or tilted for easily focusing or deflecting the ultrasonic waves.

Further, recently, researches on a phased array in which many vibrators are two-dimensionally arranged have been increasingly made. This is because a focal point of an ultrasonic beam can be formed in a desired point within a three-dimensional space by transmission of plural ultrasonic waves from a two-dimensional region. Thereby, ultrasonic image information (volume data) on a three-dimensional space within the object can be acquired, and therefore, three-dimensional images can be constructed and image quality of ultrasonic images can be improved.

However, the size of matrix type two-dimensional phased array is larger than those of other arrays (one-dimensional array and so on). Further, with microfabrication and high integration of elements, the fabrication of the two-dimensional phased array becomes difficult. Furthermore, the number of interconnections increases with increase in the number of elements, and therefore, a problem arises that a cable connected to the probe becomes thicker. It is especially difficult to apply such an array to an ultrasonic endoscope. Because the ultrasonic endoscope is inserted into a living body, and there is a strong constriction on size.

On the other hand, researches on a so-called multirow array, in which plural one-dimensional arrays are arranged in parallel, have been also made. Although the number of arrays arranged in a multirow array is not so many as that in the matrix arrangement, ultrasonic beams focused with respect to two orthogonal directions can be formed by using vibrators arranged in a two-dimensional region.

In the multirow array, the ultrasonic beam quality such as resolving power and the scanning volume remain inferior to those of the matrix arrangement array. However, the number of elements and interconnections can be drastically reduced in the multirow array, and thereby, downsizing of the ultrasonic probe and ultrasonic endoscope can be realized, and the costs can be reduced. Therefore, it is considered that there is a significant advantage in practical application of multirow array with a high performance.

Further, Japanese Patent Application Publication JP-P2000-139926A discloses an ultrasonic probe with ultrasonic transmitting and receiving means provided at a leading end of an insertion part to be inserted into a body cavity and for transmitting and receiving ultrasonic beams, and a treatment tool lead-out opening from which a treatment tool such as a puncture needle can be led out toward a scan range of ultrasonic beam by the ultrasonic transmitting and receiving means, and further, the ultrasonic probe includes ultrasonic deflecting means for deflecting the scan range of ultrasonic beam by the ultrasonic transmitting and receiving means. That is, in JP-P2000-139926A, ultrasonic vibrators are arranged in three rows and ultrasonic waves with different phases are transmitted from the respective rows for deflection of the scan range of ultrasonic waves, and thereby, the ultrasonic beam is applied to the punctuation needle even when the punctuation needle is bent.

Furthermore, Japanese Patent Application Publication JP-P2004-57460A discloses an ultrasonic diagnostic apparatus having a continuous wave Doppler mode, and the ultrasonic diagnostic apparatus includes a vibrator array having plural vibrating elements arranged in an electronic scan direction and an elevation direction perpendicular to the electronic scan direction, and a transmission and reception control unit for controlling the operation of the plural vibrating elements. In the continuous wave Doppler mode, at least one group of transmission vibrating elements arranged in the electronic scan direction and at least one group of reception vibrating elements arranged in the electronic scan direction are set in different positions from each other in the elevation direction on the vibrator array. That is, in JP-P2004-57460A, the transmission aperture and the reception aperture are taken wider by alternate arrangement of transmission vibrating element row and reception vibrating element row.

Here, a configuration of a conventional multirow array will be explained with reference to FIGS. 24-27. FIG. 24(a) is a side view showing a conventional multirow array, FIG. 24(b) is a plan view thereof, and FIG. 25 is a partially enlarged plan view thereof. Further, FIGS. 26 and 27 show interconnection methods in the multirow array.

As shown in FIGS. 24(a) and (b), the multirow array contains five element rows of L2-row, L1-row, C-row, R1-row, and R2-row in each of which 128 channels of ultrasonic transducers (also simply referred to as “elements”) are one-dimensionally arranged. Elements 901 are arranged in the C-row, elements 902 are arranged in the L1-row and the R1-row, and elements 903 are arranged in the L2-row and the R2-row. These elements are arranged on a backing layer 900. In the multirow array, the arrangement direction (X-direction) in the respective element rows is called an azimuth direction, and the direction perpendicular to the azimuth direction (Y-direction) is called an elevation direction.

As shown in FIGS. 26 and 27, each of the elements 901-903 includes a piezoelectric material layer 910 formed of lead (Pb) zirconate titanate (PZT) or the like and electrodes 911 and 912 formed on the upper face and the lower face thereof. Generally, the electrodes 912 of all elements are connected to the ground potential. Further, as shown in FIG. 24(a), an interconnections 904 to be used for supplying drive signals from the ultrasonic diagnostic apparatus main body to the elements and outputting reception signals to the ultrasonic diagnostic apparatus main body are connected to the elements 901-903, respectively.

Regarding such a multirow array, in Wildes et al., “Elevation Performance of 1.25D and 1.5D Transducer Arrays”, (IEEE TRANSACTIONS ON ULTRASONICS, FERROELECTRICS, AND FREQUENCY CONTROL, VOL. 44, NO. 5, SEPTEMBER 1997, pp. 1027-1037), the performance of multirow array is studied, in which the vibrator arrangement in the elevation direction and the interconnection method are changed. Here, according to the definition of Wildes et al., the dimensions of arrays can be explained as in the following (1) to (5).
(1) 1D array: Plural elements are arranged in one row (in the azimuth direction). Accordingly, the aperture diameter in the elevation direction (the element width in this case) is fixed, and the focal point of ultrasonic beam is formed by an acoustic lens or the like, and the focal length is fixed.
(2) 1.25D array: As shown in FIG. 26, plural elements are arranged in several rows (five rows in FIG. 26). The elements arranged in positions symmetric with respect to a central axis in the longitudinal direction of the array are connected in parallel (connected to a common interconnection). Since the plural rows of elements are arranged in the elevation direction, the aperture diameter can be changed. Note that the focal point of ultrasonic beam is formed by an acoustic lens or the like, and the focal length is fixed.
(3) 1.5D array: Plural elements are arranged in several rows as is the case of the 1.25D array. The elements arranged in positions symmetric with respect to a central axis in the longitudinal direction of the array are connected in parallel, and further, the focal point of ultrasonic beam is formed by electronic control. Accordingly, the focal point of ultrasonic beam can be dynamically changed. Note that the elements are symmetrically and commonly interconnected, and thereby, the ultrasonic beam cannot be deflected in the elevation direction.
(4) 1.75D array: Plural elements are arranged in several rows, and further, the respective elements are independently interconnected. That is, as shown in FIG. 27, the plural elements arranged in the elevation direction are separately interconnected. Thereby, the ultrasonic beam can be deflected in the elevation direction in addition to dynamical change of the aperture diameter and the focal length of ultrasonic beam although the number of interconnections is larger than that of the 1.5D array.
(5) 2D array: Plural elements are arranged in substantially the same number in both the elevation direction and the azimuth direction to form a matrix. Accordingly, apodization, the focal length of ultrasonic beam in the three-dimensional space, and the transmission direction (deflection) of ultrasonic beam can be electrically controlled.

As to the 1.25D array, the 1.5D array, and the 1.75D array, since the degrees of freedom of control of ultrasonic beam are between those of the 1D array and 2D array, they are called the 1.25D array, the 1.5D array, and the 1.75D array.

In such a multirow array, in order to improve the quality of ultrasonic beam by reducing the grating lobes, the arrangement pitch of elements in the azimuth direction is typically set to equal to one another to or less than the wavelength of transmission ultrasonic waves. Assuming that the sound speed in the living body is about 1500 m/s, the wavelength of ultrasonic waves at a frequency of 5 MHz is about 300 μm. In FIGS. 24 and 25, the arrangement pitch in the azimuth direction is 150 μm.

On the other hand, as shown in FIGS. 24 and 25, with respect to the elevation direction, the widths W₁ to W₃ of the elements 901-903 become narrower from inside (C-row) to outside (L2-row and R2-row). Such arrangement is for improving the quality of ultrasonic beam, and arrangement methods called Fresnel arrangement, MIAE (minimum integrated absolute time-delay error) arrangement, and so on are known. In the multirow array shown in FIGS. 24 and 25, the Fresnel arrangement is adopted, and the width W₁ of the element 901 of the C-row is 6.9 mm, the width W₂ of the element 902 of the L1-row and the R1-row is 1.4 mm, and the width W₃ of the element 903 of the L2-row and the R2-row is 1.1 mm.

Refer to Wildes et al., which is incorporated herein by reference, for details of the Fresnel arrangement and MIAE arrangement.

As shown in FIG. 25, since the widths W₁ to W₃ of the elements 901-903 are different from one another while the lengths X₁ to X₃ of the elements 901-903 are common, the electric impedance values vary with respect to each row. Accordingly, degrees of electric impedance matching between the ultrasonic diagnostic apparatus main body and themselves are different, and thus, there are disadvantages that the sensitivity of the elements in transmission and reception of ultrasonic beams vary with respect to each row and the frequency characteristic as a system varies.

On the other hand, Japanese Utility Model Application Publication JP-U-5-9514 discloses an ultrasonic two-dimensional array probe in which the numbers of elements are different in a central part and an end part, and the probe is configured by elements made of a piezoelectric material having relative permittivity ε₃₃ in the thickness direction that is nearly inversely proportional to the ratio of lengths in the focus direction in a Fresnel pattern degenerated to one-dimension. That is, in JP-U-5-9514, even when the numbers of central elements and end elements are different, plural kinds of PZT having different properties are provided according to the segments (see FIG. 1 of JP-U-5-9514) in order to obtain electrical matching and good transmission and reception characteristic.

However, since it is required to prepare plural kinds of PZT for manufacturing one ultrasonic transducer array, the manufacturing process becomes complex and the manufacturing cost rises. Further, as described in the paragraph 0012 of JP-A-7-203592, it is not preferable to prepare plural kinds of PZT because frequency characteristics are different if the kinds of PZT are different.

### SUMMARY OF THE INVENTION

Accordingly, in view of the above-mentioned points, a purpose of the present invention is, in an ultrasonic transducer array in which plural kinds of ultrasonic transducers having different shapes from one another are arranged, to make electric impedances among the ultrasonic transducers equal to one another to one another.

In order to achieve the above-mentioned purpose, an ultrasonic transducer array according to a first aspect of the present invention is an ultrasonic transducer array in which plural ultrasonic transducers having at least two kinds of shapes are arranged, and the ultrasonic transducer array includes: a first ultrasonic transducer; and a second ultrasonic transducer having a different shape from that of the first ultrasonic transducer, and an area of an ultrasonic transmission/reception face of the second ultrasonic transducer is substantially equal to one another to an area of an ultrasonic transmission/reception face of the first ultrasonic transducer.

Further, an ultrasonic transducer array according to a second aspect of the present invention is an ultrasonic transducer array in which plural ultrasonic transducers are arranged, and the ultrasonic transducer array includes: at least one first ultrasonic transducer commonly connected to a first interconnection; and plural second ultrasonic transducers commonly connected to a second interconnection, and a sum of areas of ultrasonic transmission/reception faces of the plural second ultrasonic transducers is substantially equal to one another to a sum of areas of ultrasonic transmission/reception faces of the at least one first ultrasonic transducer.

In this application, the expression that areas or electric impedances are substantially equal includes not only the case where values in comparison are strictly equal but also the case where they are equal within a predetermine range of error, that is, those values are generally equal. For example, when the difference between the values in comparison is within ±10%, it can be said that they are substantially equal to one another.

Further, the expression that different in shapes means that two figures in comparison have other relationships than congruity or similarity, and includes the case where the horizontal to vertical ratios of rectangular shapes are different and the case where the ratios of outer circumference diameter to inner circumference diameter of annular shapes are different.

According to the present invention, since the areas of ultrasonic transmission/reception faces of plural kinds of ultrasonic transducers having different shapes are made substantially equal to one another, the values of electric impedances of the ultrasonic transducers can be made equal among the plural kinds of ultrasonic transducers. Thereby, degrees of electric impedance matching between the ultrasonic diagnostic apparatus main body and themselves are made equal to one another. As a result, transmission sensitivity and reception sensitivity of ultrasonic waves can be made equal. Therefore, by using an ultrasonic probe or ultrasonic endoscope including such an ultrasonic transducer array, the quality of ultrasonic beams to be transmitted can be improved and good quality ultrasonic images can be easily generated based on the acquired reception signals in the ultrasonic diagnostic apparatus.

### BRIEF DESCRIPTION OF THE DRAWINGS

FIG. 1 shows a configuration of an ultrasonic transducer array according to the first embodiment of the present invention;

FIG. 2 shows a configuration of an ultrasonic probe according to one embodiment of the present invention;

FIG. 3 shows a configuration of an ultrasonic diagnostic apparatus according to one embodiment of the present invention;

FIG. 4 is a partially enlarged plan view of the ultrasonic transducer array shown in FIG. 1;

FIG. 5 shows a configuration of an ultrasonic transducer array according to the second embodiment of the present invention;

FIG. 6 is a partially enlarged plan view of the ultrasonic transducer array shown in FIG. 5;

FIG. 7 shows a configuration of an ultrasonic transducer array according to the third embodiment of the present invention;

FIG. 8 is a partially enlarged plan view of the ultrasonic transducer array shown in FIG. 7;

FIG. 9 shows a configuration of an ultrasonic transducer array according to the fourth embodiment of the present invention;

FIG. 10 is a partially enlarged plan view of the ultrasonic transducer array shown in FIG. 9;

FIG. 11 shows a configuration of an ultrasonic transducer array according to the fifth embodiment of the present invention;

FIG. 12 is a partially enlarged plan view of the ultrasonic transducer array shown in FIG. 11;

FIG. 13 shows a configuration of an ultrasonic transducer array according to the sixth embodiment of the present invention;

FIG. 14 is a partially enlarged plan view of the ultrasonic transducer array shown in FIG. 13;

FIG. 15 shows a configuration of an ultrasonic transducer array according to the seventh embodiment of the present invention;

FIG. 16 is a partially enlarged plan view of the ultrasonic transducer array shown in FIG. 15;

FIG. 17 shows a configuration of an ultrasonic transducer array according to the eighth embodiment of the present invention;

FIG. 18 is a partially enlarged plan view of the ultrasonic transducer array shown in FIG. 17;

FIG. 19 is a schematic diagram showing a configuration of an ultrasonic endoscope according to one embodiment of the present invention;

FIG. 20 is an enlarged schematic diagram showing the leading end of an insertion part shown in FIG. 19;

FIG. 21 is a plan view showing an example of an ultrasonic transducer array in which plural kinds of elements are asymmetrically arranged;

FIG. 22 is a plan view showing an example of an ultrasonic transducer array in which plural kinds of oval elements are arranged;

FIG. 23 is a plan view showing an example of an ultrasonic transducer array in which plural kinds of polygonal elements are arranged;

FIG. 24 shows a configuration of a conventional multirow array;

FIG. 25 is a partially enlarged plan view of the ultrasonic transducer array shown in FIG. 24;

FIG. 26 is a side view showing arrangement and connection methods of elements in 1.25D array or 1.5D array; and

FIG. 27 is a side view showing arrangement and connection methods of elements in 1.75D array.

### DESCRIPTION OF THE PREFERRED EMBODIMENTS

Hereinafter, preferred embodiments of the present invention will be explained in detail with reference to the drawings. The same reference numerals will be assigned to the same component elements and the description thereof will be omitted.

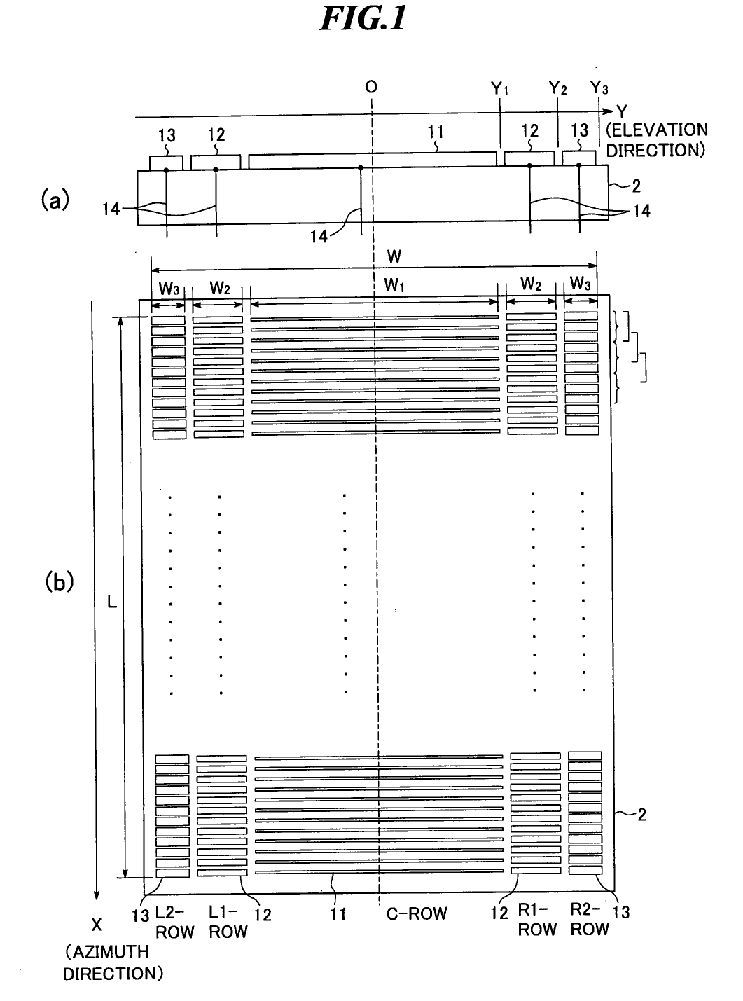

FIG. 1 schematically shows an ultrasonic transducer array according to the first embodiment of the present invention. FIG. 1(a) is a side view showing a state in which the ultrasonic transducer array according to the embodiment is provided on a backing layer, and FIG. 1(b) is a plan view thereof.

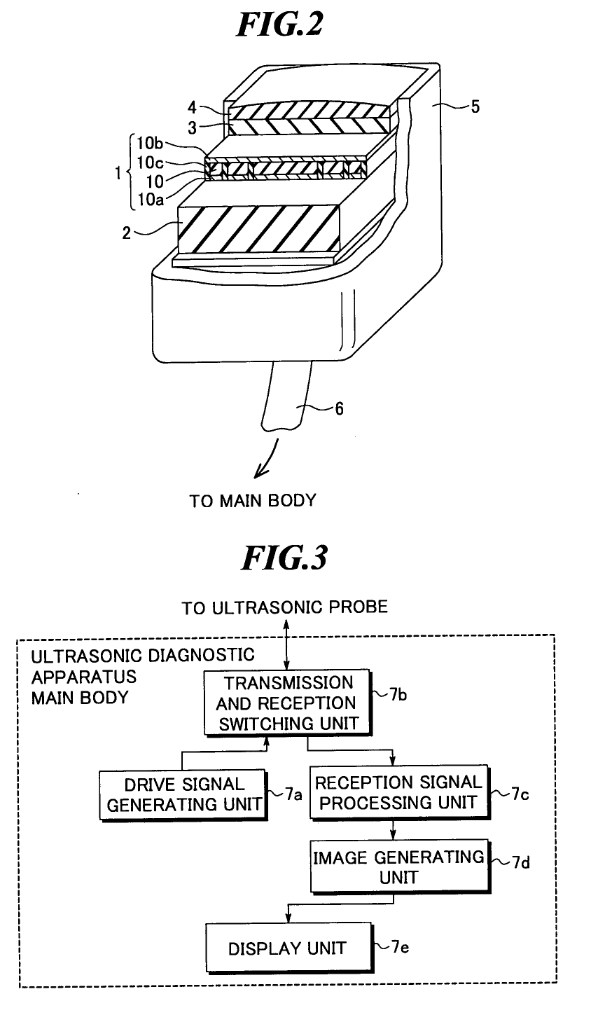

Further, FIG. 2 is a partially sectional perspective view showing an ultrasonic probe according to one embodiment of the present invention, and FIG. 3 is a block diagram showing an ultrasonic diagnostic apparatus according to one embodiment of the present invention. The ultrasonic transducer array shown in FIG. 1 is provided for use in the ultrasonic probe as shown in FIG. 2, an ultrasonic endoscope, which will be described later, and so on.

As shown in FIG. 2, the ultrasonic probe according to the embodiment includes an ultrasonic transducer array 1, a backing layer 2, and an acoustic matching layer 3. Further, the ultrasonic probe may include an acoustic lens 4 according to need. These parts are accommodated in a casing 5. Further, interconnections drawn from the ultrasonic transducer array 1 are connected to an electronic circuit contained in an ultrasonic imaging apparatus man body via a cable 6.

The ultrasonic transducer array 1 includes plural ultrasonic transducers that expand and contract to generate ultrasonic waves when drive signals are supplied thereto, and receive ultrasonic waves propagating from an object to be inspected and output electric signals (reception signals). Each ultrasonic transducer is formed of a vibrator including a piezoelectric material 10 such as PZT (lead (Pb) zirconate titanate) having electrodes 10a and 10b provided on both ends thereof. Either one of the electrodes 10a and 10b may be formed continuously over plural vibrators. In FIG. 2, the electrode 10b on the upper faced of the vibrators is a common electrode (ground electrode).

Further, a filler 10c such as urethane resin or epoxy resin may be provided between and around the plural vibrators for protection of the respective vibrators and suppression of unwanted propagation of ultrasonic waves (e.g., propagation of ultrasonic waves within the arrangement surface of the vibrators). As below, one ultrasonic transducer is also simply referred to as “element”.

The backing layer 2 is formed of a material having large acoustic attenuation such as an epoxy resin containing ferrite powder, metal powder, or PZT powder, or rubber containing ferrite powder, and promotes attenuation of unwanted ultrasonic waves generated from the ultrasonic transducer array 1.

The acoustic matching layer 3 is formed of, for example, Pyrex (registered trademark) glass or an epoxy resin containing metal powder, which easily propagates ultrasonic waves, and resolves the mismatch of acoustic impedances between the object such as a living body and the ultrasonic transducer. Thereby, the ultrasonic waves transmitted from the ultrasonic transducers efficiently propagate within the object.

The acoustic lens 4 is formed of silicon rubber, for example, and focuses an ultrasonic beam transmitted from the ultrasonic transducer array 1 and propagating the acoustic matching layer 3 at a predetermined depth within the object. In the ultrasonic transducer array according to the embodiment as explained below, the acoustic lens 4 is not necessarily provided because the ultrasonic beam can be focused by electronic control, however, the acoustic lens 4 may be simultaneously used for improvement in the focus effect of ultrasonic beam.

As shown in FIG. 3, the ultrasonic diagnostic apparatus main body includes a drive signal generating unit 7a for generating drive signals to be respectively supplied to the elements arranged in the ultrasonic transducer array 1 of the ultrasonic probe, a transmission and reception switching unit 7b for switching between the output of drive signals to the ultrasonic probe and the input of reception signals from the ultrasonic probe, a reception signal processing unit 7c for performing predetermined signal processing such as amplification, phase adjustment and signal addition, and detection on the reception signals outputted from the respective elements of the ultrasonic probe, an image generating unit 7d for generating ultrasonic images based on the reception signals that have been subjected to the predetermined signal processing, and a display unit 7e for displaying the ultrasonic images.

With reference to FIG. 1 again, the ultrasonic transducer array according to the embodiment includes three kinds of elements 11-13 arranged in five rows of L2-row, L1-row, C-row, R1-row, and R2-row. These elements 11-13 are arranged at intervals of 30 μm between the elements located in adjacent rows and themselves. The entire width W of the ultrasonic transducer array (the size in the Y-axis direction) is 5 mm and the length L (the size in the X-axis direction) is 19 mm. Hereinafter, the Y-axis direction is also referred to as “elevation direction” and the X-axis direction is also referred to as “azimuth direction”.

In each row, 128 channels of ultrasonic transducers (elements) are arranged. The arrangement pitch P of these elements is commonly 150 μm in each row. The arrangement pitch P is designed such that the pitch is equal to or less than the half of the wavelength of transmission ultrasonic waves in consideration of generation angle of the grating lobes in the electronic sector scan system. For example, when the frequency of the transmission ultrasonic waves is 5 MHz, assuming that the sound speed in the living body is about 1500 m/s, the half of the wavelength of the ultrasonic waves is 150 μm.

Further, as shown in FIG. 1(a), the respective elements 11-13 arranged in each row are independently connected to interconnections 14. Drive signals generated in the ultrasonic diagnostic apparatus main body are respectively supplied to the elements 11-13 via the interconnections 14.

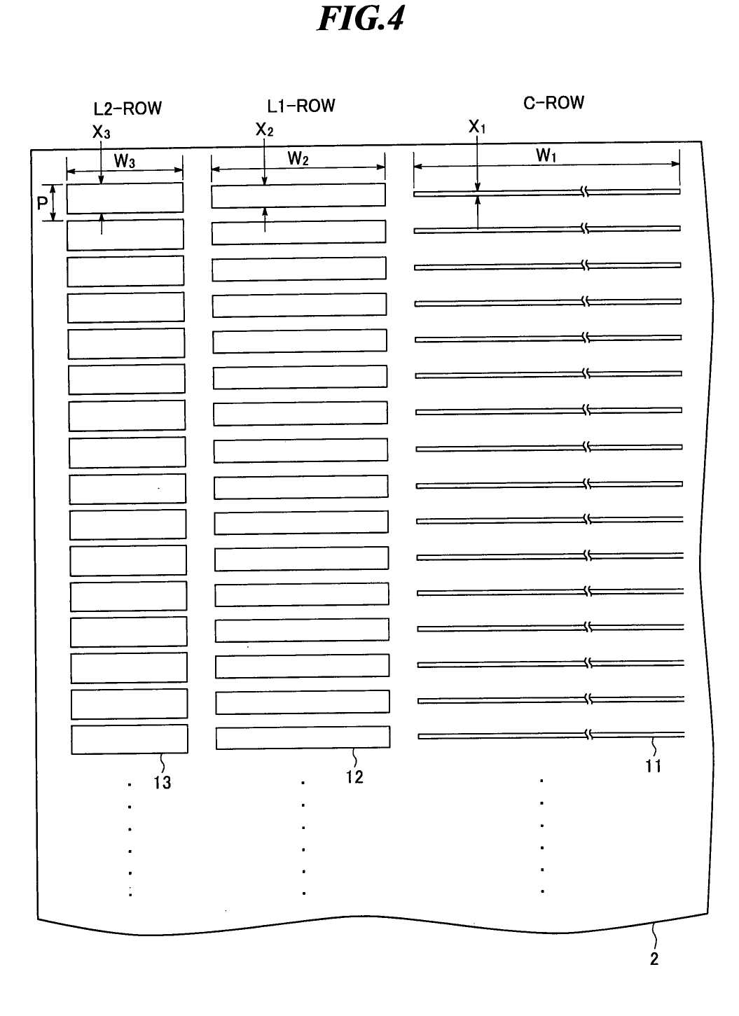

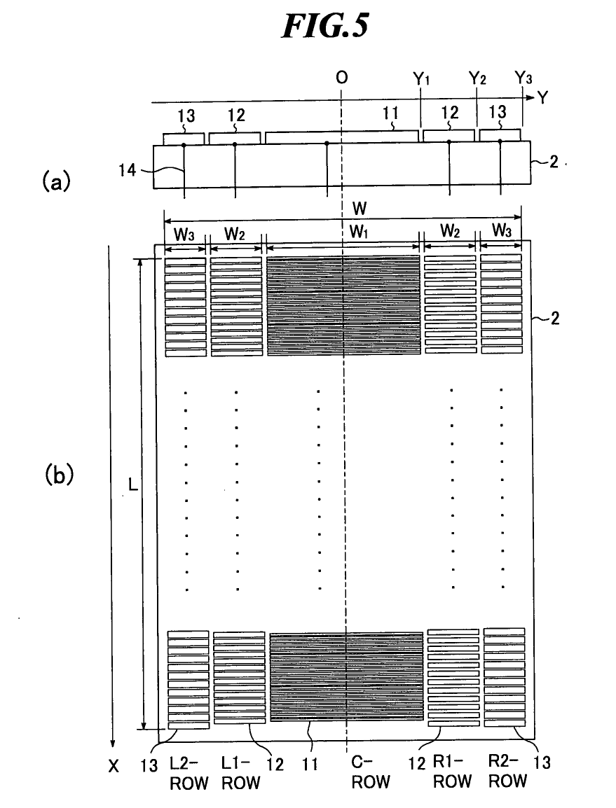

FIG. 4 is a partially enlarged plan view of the ultrasonic transducer array 1 shown in FIG. 1. The widths W₁ to W₃ and positions Y₁ to Y₃ of the elements 11-13 (FIG. 1) are designed in the Fresnel arrangement. The Fresnel arrangement is one of the arrangement methods by which the widths W₁ to W₃ of the elements become narrower toward outside from the center in the elevation direction. Further, the lengths X₁ to X₃ of the elements 11-13 are designed such that areas S₁-S₃ of ultrasonic transmission/reception faces are equal to one another according to the widths W₁ to W₃ of the elements 11-13.

With reference to FIG. 4, regarding the size of the element 11 arranged in the central C-row, the width (the size in the elevation direction) W₁ is 2880 μm and the length (the size in the azimuth direction) X₁ is 17.9 μm. Therefore, the area S₁ of the element 11 is obtained by S₁=W₁×X₁=51552≈51600 (μm²). The element interval in the C-row is 132.1 μm (arrangement pitch 150 μm−element length 17.9 μm).

Further, regarding the size of the element 12 arranged in the L1-row and R1-row at both adjacent sides of the C-row, the width W₂ is 570 μm and the length X₂ is 90.5 μm. Therefore, the area S₂ of the element 12 is obtained by S₂=W₂×X₂=51585≈51600 (μm²) The element interval in the L1-row and R1-row is 59.5 μm (arrangement pitch 150 μm−element length 90.5 μm).

Furthermore, regarding the size of the element 13 arranged in the L2-row and R2-row outside of the L1-row and R1-row, the width W₃ is 430 μm and the length X₃ is 120 μm. Therefore, the area S₃ of the element 13 is obtained by S₃=W₃×X₃=51600 (μm²). The element interval in the L2-row and R2-row is 30 μm (arrangement pitch 150 μm−element length 120 μm)

When ultrasonic waves are transmitted by using such an ultrasonic transducer array, the elements 11-13 contained in one or some columns (e.g., three to five columns) arranged along the elevation direction are set as one set of drive elements to be simultaneously used, and the elements are driven by the drive signals while predetermined delay times are provided to the drive signals. Thereby, an ultrasonic beam having a focal point at a desired depth can be transmitted in a desired direction.

Further, the drive elements are sequentially driven while the set positions of the set of drive elements are displaced in the azimuth direction, and thus, the ultrasonic beam can be scanned by electronic control. In this regard, the present set of drive elements may be set such that the elements are not overlapped with the previous set of drive elements. For example, the elements of the first to third columns are used in the first transmission, the elements of the fourth to sixth columns are used in the second transmission, and the elements of the seventh to ninth columns are used in the third transmission.

Alternatively, the present set of drive elements may be set such that the elements are partly overlapped with the previous set of drive elements. For example, the elements of the first to third columns are used in the first transmission, the elements of the third to fifth columns are used in the second transmission, and the elements of the fifth to seventh columns are used in the third transmission.

As described above, according to the embodiment, although the elements 11-13 have different widths from one another, the lengths of the respective elements are defined such that the areas thereof are equal to one another, and thus, the electric impedances thereof are nearly equal to one another. Thereby, matching characteristics of electric impedances with the ultrasonic diagnostic apparatus main body (see FIG. 3) can be made equal among the elements, and consequently, the characteristics such as transmission sensitivity and reception sensitivity of ultrasonic waves can be made equal to one another. Therefore, when generating ultrasonic images based on the reception signals received by the elements 11-13, image quality of the ultrasonic images can be improved.

Further, according to the embodiment, since the interconnections 14 are independently connected to the respective elements 11-13, the supply timing of drive signals to the elements set as drive elements is controlled, and thereby, not only the focal length can be changed but also the ultrasonic beam can be deflected in the elevation direction. Therefore, ultrasonic image information (volume data) on the respective positions in the three-dimensional space within the object can be acquired without change in the position and orientation of the ultrasonic probe, and thus, three-dimensional images can be constructed at a high speed.

Furthermore, according to the embodiment, since the element intervals in the azimuth direction can be taken wider especially in the L1-row, C-row, and R1-row, the crosstalk of ultrasonic waves can be reduced. Therefore, the contrast can be improved in ultrasonic images generated based on the reception signal outputted from the elements.

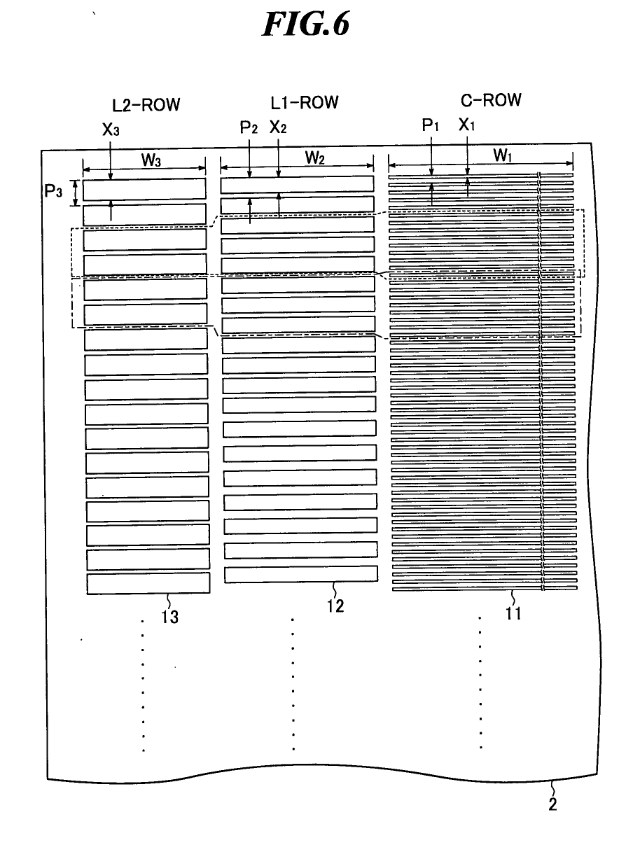

Next, an ultrasonic transducer array according to the second embodiment of the present invention will be explained with reference to FIGS. 5 and 6. FIG. 5(a) is a side view showing a state in which the ultrasonic transducer array according to the embodiment is provided on a backing layer, FIG. 5(b) is a plan view thereof, and FIG. 6 is a partially enlarged plan view showing ultrasonic transducers shown in FIG. 5(b).

In the ultrasonic transducer array according to the embodiment, the arrangement of elements is changed from that of the ultrasonic transducer array shown in FIG. 1. That is, the element intervals in the azimuth direction are unified to 30 μm in the C-row where the elements 11 are arranged, in the L1-row and the R1-row where the elements 12 are arranged, and in the L2-row and the R2-row where the elements 13 are arranged. The element interval of 30 μm is designed such that the arrangement pitches P1-P3 of the elements containing the lengths X₁ to X₃ of the elements 11-13 are equal to or less than the half of the wavelength of the transmission ultrasonic waves (e.g., 150 μm or less) at the maximum.

The sizes of the respective elements 11-13, the arrangement in the elevation direction (Fresnel arrangement), and the interconnection method (independent interconnection) are the same as those in the first embodiment. Further, the entire width W of the ultrasonic transducer array is, for example, 5 mm and the length L is, for example, 5 mm.

Specifically, with reference to FIG. 6, 400 channels of elements 11 are arranged at the arrangement pitch P₁=47.9 μm (element 11 length 17.9 μm+element interval 30 μm) in the central C-row. Further, 159 channels of elements 12 are arranged at the arrangement pitch P₂=120.5 μm (element 12 length 90.5 μm+element interval 30 μm) in the L1-row and R1-row. Furthermore, 128 channels of elements 13 are arranged at the arrangement pitch P₃=150 μm (element 13 length 120 μm+element interval 30 μm) in the L2-row and R2-row.

When ultrasonic waves are transmitted by using such an ultrasonic transducer array, the elements 11-13 contained in a predetermined range are set as one set of drive elements to be simultaneously used, and the elements are driven while predetermined delay times are provided to the elements. Thereby, an ultrasonic beam having a focal point at a desired depth can be transmitted in a desired direction. For example, as shown by the dashed line in FIG. 6, at the time of Nth transmission, the elements 13 contained in the third and fourth columns of the L2-row and R2-row, the elements 12 contained in the third to fifth columns of the L1-row and R1-row, and the elements 11 contained in the sixth to fourteenth columns of the C-row are set as drive elements. Further, when the next transmission is performed, the present set of drive elements may be set such that the elements are not overlapped with the previous set of drive elements, or the present set of drive elements may be set such that the elements are partly overlapped with the previous set of drive elements as shown by the dashed-dotted line.

As described above, according to the embodiment, since the density of elements is made higher toward the center from outside in the elevation direction, the filling ratio of elements to the entire area of the ultrasonic transducer array can be improved. Thereby, the transmission and reception sensitivity of ultrasonic waves and resolving power can be improved, and therefore, image quality of ultrasonic images generated in the ultrasonic diagnostic apparatus can be improved.

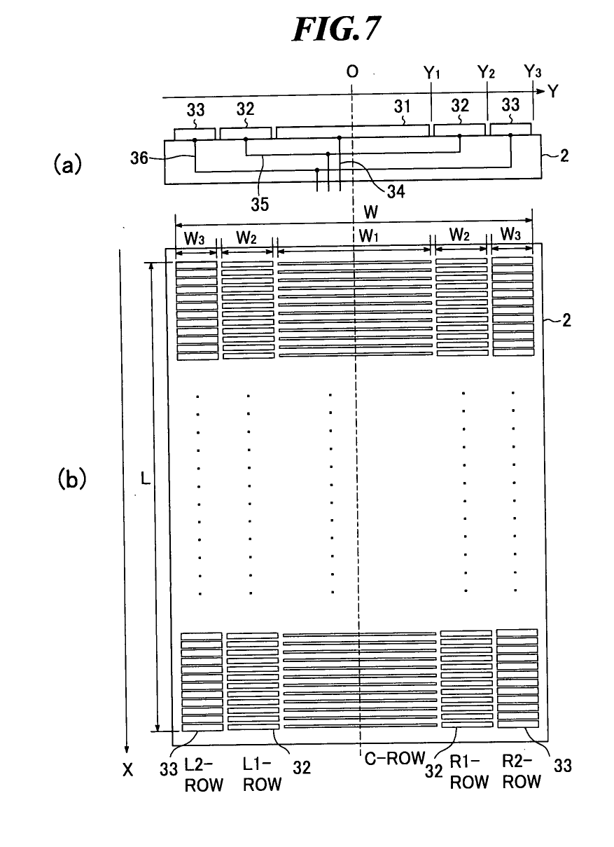

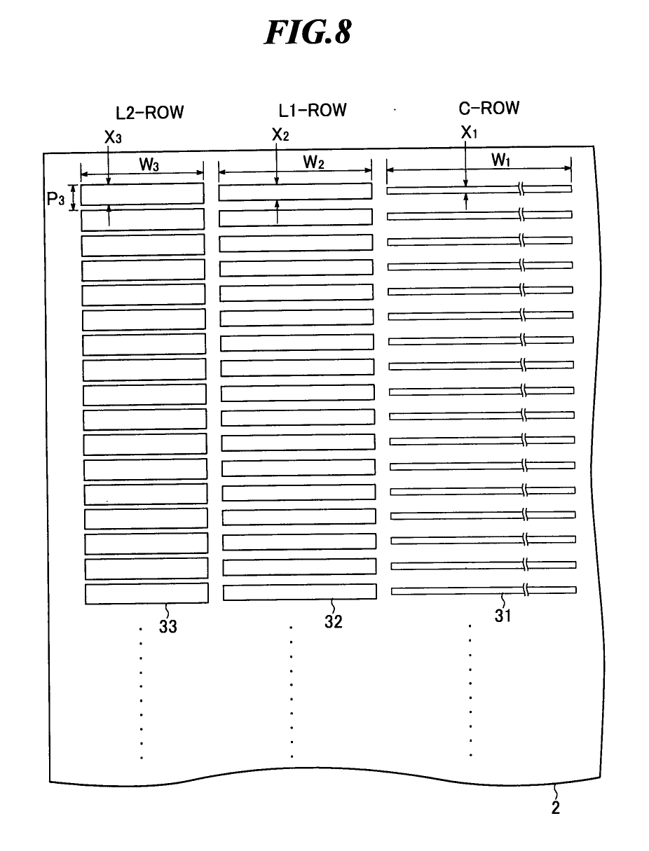

Next, an ultrasonic transducer array according to the third embodiment of the present invention will be explained with reference to FIGS. 7 and 8. FIG. 7(a) is a side view showing a state in which the ultrasonic transducer array according to the embodiment is provided on a backing layer, FIG. 7(b) is a plan view thereof, and FIG. 8 is a partially enlarged plan view showing ultrasonic transducers shown in FIG. 7(b).

In the ultrasonic transducer array according to the embodiment, the interconnection method and element size are changed from those of the ultrasonic transducer array shown in FIG. 1.

As shown in FIG. 7(b), the ultrasonic transducer array includes three kinds of elements 31-33 arranged in five rows of L2-row, L1-row, C-row, R1-row, and R2-row. These elements 31-33 are arranged at intervals of 30 μm between the elements arranged in adjacent rows and themselves. The entire width W of the ultrasonic transducer array is 5mm and the length L is 19 mm.

In each row, 128 channels of elements 31-33 are arranged. The arrangement pitch P of these elements is 150 μm that is equal to or less than the half of the wavelength of the transmission ultrasonic waves.

Further, as shown in FIG. 7(a), the elements in the C-row are connected to interconnections 34, the elements in the L1-row and R1-row are connected to interconnections 35, and the elements in the L2-row and R2-row are connected to interconnections 36. Thus, the elements arranged symmetrically with respect to the center in the elevation direction are commonly interconnected, and thereby, drive signals generated in the ultrasonic diagnostic apparatus main body are supplied to those elements with the same timing.

With reference to FIGS. 7 and 8, the widths W₁ to W₃ and positions Y₁ to Y₃ of the elements 31-33 are designed in the Fresnel arrangement. Further, the lengths X₁ to X₃ of the elements 31-33 are designed such that the sums of areas of ultrasonic transmission/reception faces with respect to each interconnection are generally equal to one another according to the widths W₁ to W₃ of the elements 31-33.

Specifically, regarding the size of the element 31 arranged in the C-row, the width W₁ is 2880 μm and the length X₁ is 35.8 μm. Therefore, the area S₁ of the element 31 is obtained by S₁ =W₁×X₁=103104≈103200 (μm²). The element interval in the C-row is 114.2 μm (arrangement pitch 150 μm−element length 35.8 μm).

Further, regarding each size of the two elements 32 arranged in the L1-row and R1-row and commonly interconnected, the width W₂ is 570 μm and the length X₂ is 90.5 μm. Therefore, the sum of areas S₂ (L+R) of the elements 32 is obtained by S_{2(L+R)}=W₂×X₂×2=103170≈103200 (μm²) . The element interval in the L1-row and R1-row is 59.5 μm (arrangement pitch 150 μm−element length 90.5 μm).

Furthermore, regarding each size of the two elements 33 arranged in the L2-row and R2-row and commonly interconnected, the width W₃ is 430 μm and the length X₃ is 120 μm. Therefore, the sum of areas S_{3(L+R)} of the elements 33 is obtained by S_{3(L+R)}=W₃×X₃×2=103200 (μm²). The element interval in the L2-row and R2-row is 30 μm (arrangement pitch 150 μm−element length 120 μm).

As described above, according to the embodiment, since areas of the respective elements are designed such that the total areas of the commonly interconnected elements (one element for the C-row) are generally equal to one another, the synthesized electric impedances with respect to each interconnection are nearly equal to one another, and the characteristics thereof are substantially the same. Thereby, characteristics of transmission sensitivity and reception sensitivity of ultrasonic waves can be made equal to one another, and image quality of the ultrasonic images generated in the ultrasonic diagnostic apparatus main body can be improved. Further, in the embodiment, the number of interconnections can be reduced by commonly interconnecting plural elements, and therefore, the downsizing of the ultrasonic probe, the thinning down of the cable, and the reduction in costs can be realized.

Furthermore, according to the embodiment, since the element intervals in the azimuth direction can be taken wider especially in the L1-row, C-row, and R1-row, the crosstalk of ultrasonic waves can be reduced. Thereby, the contrast can be improved in ultrasonic images.

The method of setting the drive elements to be used when ultrasonic waves are transmitted is the same as that explained in the first embodiment. Further, in the embodiment, the focal length of the ultrasonic beam transmitted in a substantially perpendicular direction to the ultrasonic transmission/reception face can be changed by adjustment of drive timing among the one set of elements that have been set as drive elements. Therefore, ultrasonic images with higher resolving power can be generated by dynamic focus than in the case of using a 1D array.

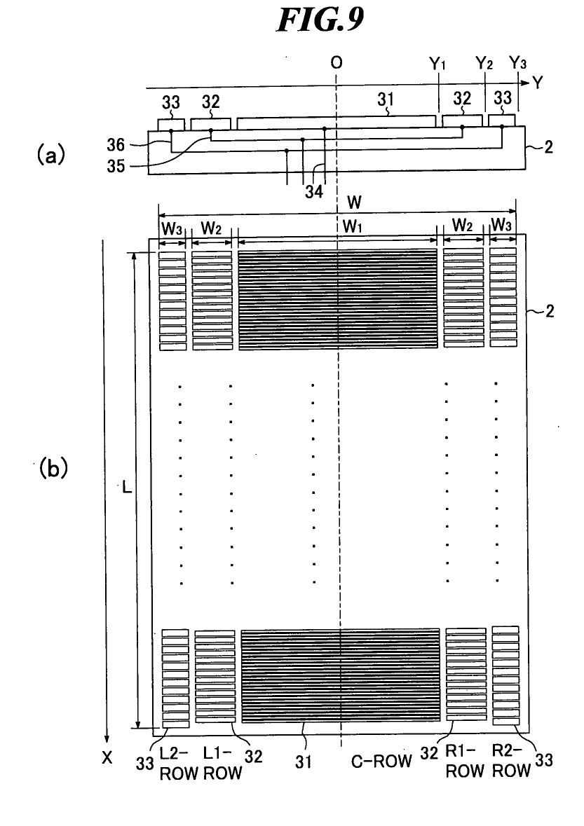

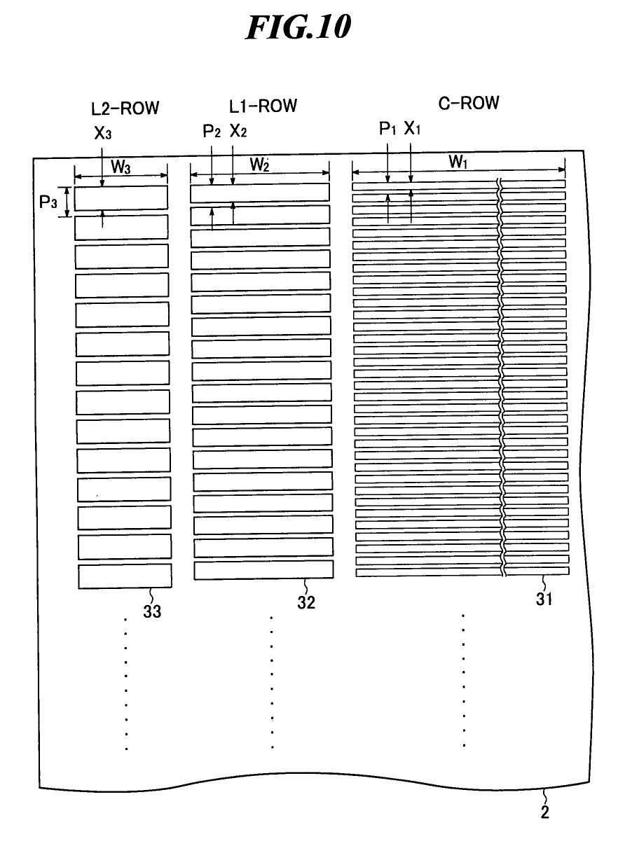

Next, an ultrasonic transducer array according to the fourth embodiment of the present invention will be explained with reference to FIGS. 9 and 10. FIG. 9(a) is a side view showing a state in which the ultrasonic transducer array according to the embodiment is provided on a backing layer, FIG. 9(b) is a plan view thereof, and FIG. 10 is a partially enlarged plan view showing ultrasonic transducers shown in FIG. 9(b).

In the ultrasonic transducer array according to the embodiment, the arrangement of elements is changed from that of the ultrasonic transducer array shown in FIG. 7. That is, the element intervals in the azimuth direction are unified to 30 μm in the C-row where the elements 31 are arranged, in the L1-row and the R1-row where the elements 32 are arranged, and in the L2-row and the R2-row where the elements 33 are arranged. The element interval of 30 μm is designed such that the arrangement pitches P1-P3 of the elements containing the lengths X₁ to X₃ of the elements 31-33 are equal to or less than the half of the wavelength of the transmission ultrasonic waves (e.g., 150 μm or less) at the maximum.

The sizes of the respective elements 31-33, the arrangement in the elevation direction (Fresnel arrangement), and the interconnection method (common interconnection) are the same as those in the third embodiment. Further, the entire width W of the ultrasonic transducer array is, for example, 5 mm and the length L is, for example, 5 mm.

Specifically, with reference to FIG. 10, 292 channels of elements 31 are arranged at the arrangement pitch P₁=65.8 μm (element length 35.8 μm+element interval 30 μm) in the central C-row. Further, 159 channels of elements 32 are arranged at the arrangement pitch P₂=120.5 μm (element length 90.5 μm+element interval 30 μm) in the L1-row and R1-row. Furthermore, 128 channels of elements 33 are arranged at the arrangement pitch P₃=150 μm (element length 120 μm+element interval 30 μm) in the L2-row and R2-row.

As described above, according to the embodiment, since the density of elements is made higher toward the center from outside in the elevation direction, the filling ratio of elements to the entire area of the ultrasonic transducer array can be improved. Thereby, the transmission and reception sensitivity of ultrasonic waves and resolving power can be improved, and therefore, image quality of ultrasonic images generated in the ultrasonic diagnostic apparatus main body can be improved. Further, the number of interconnections can be reduced by using common interconnections, and therefore, the downsizing of the ultrasonic probe, the thinning down of the cable, and the reduction in costs can be realized.

The method of setting the drive elements to be used when ultrasonic waves are transmitted is the same as that explained in the second embodiment.

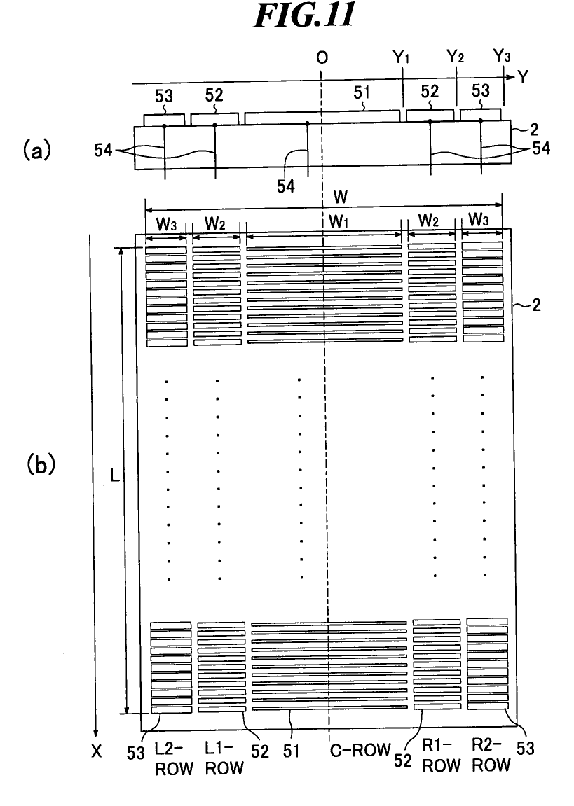

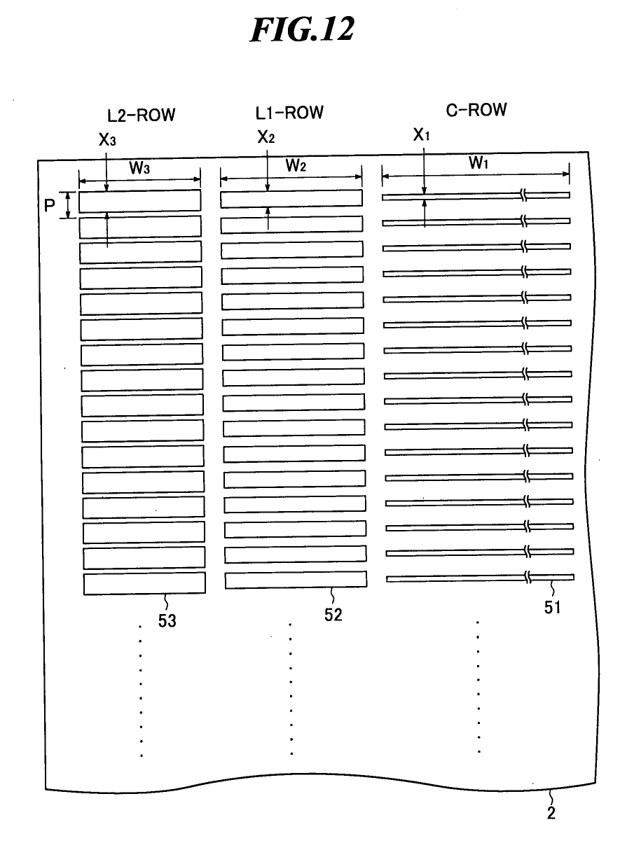

Next, an ultrasonic transducer array according to the fifth embodiment of the present invention will be explained with reference to FIGS. 11 and 12. FIG. 11(a) is a side view showing a state in which the ultrasonic transducer array according to the embodiment is provided on a backing layer, FIG. 11(b) is a plan view thereof, and FIG. 12 is a partially enlarged plan view showing ultrasonic transducers shown in FIG. 11(b).

In the ultrasonic transducer array according to the embodiment, the arrangement of the elements in the elevation direction are changed from that of the ultrasonic transducer array shown in FIG. 1.

As shown in FIG. 11, the ultrasonic transducer array includes three kinds of elements 51-53 arranged in five rows of L2-row, L1-row, C-row, R1-row, and R2-row. These elements 51-53 are arranged at intervals of 30 μm between the elements arranged in adjacent rows and themselves. The entire width W of the ultrasonic transducer array is 5 mm and the length L is 19 mm.

In each row, 128 channels of ultrasonic transducers (elements) are arranged. The arrangement pitch P in the respective rows is 150 μm that is equal to or less than the half of the wavelength of the transmission ultrasonic waves.

Further, as shown in FIG. 11(a), the respective elements 51-53 are independently connected to interconnections 54.

With reference to FIGS. 11 and 12, the widths W₁ to W₃ and positions Y₁ to Y₃ of the elements 51-53 are designed in the MIAE (minimum integrated absolute time-delay error) arrangement. The MIAE arrangement is one of the arrangement methods by which the widths W₁ to W₃ of the elements become narrower toward outside from the center in the elevation direction. Further, the lengths X₁ to X₃ of the elements 51-53 are designed such that the areas S₁-S₃ of ultrasonic transmission/reception faces are equal to one another according to the widths W₁ to W₃ of the elements 11-13.

Specifically, regarding the size of the element 51 arranged in the C-row, the width W₁ is 2290 μm and the length X₁ is 30.7 μm. Therefore, the area S₁ of the element 51 is obtained by S₁=W₁×X₁=70303≈70200 (μm²). The element interval in the C-row is 119.3 μm (arrangement pitch 150 μm−element length 30.7 μm).

Further, regarding the size of the element 52 arranged in the L1-row and R1-row, the width W₂ is 710 μm and the length X₂ is 98.9 μm. Therefore, the area S₂ of the element 52 is obtained by S₂=W₂×X₂=70219≈70200 (μm²). The element interval in the L1-row and R1-row is 51.1 μm (arrangement pitch 150 μm−element length 98.9 μm).

Furthermore, regarding the size of the element 53 arranged in the L2-row and R2-row, the width W₃ is 585 μm and the length X₃ is 120 μm. Therefore, the area S₃ of the element 53 is obtained by S₃=W₃×X₃=70200 (μm²). The element interval in the L2-row and R2-row is 30 μm (arrangement pitch 150 μm−element length 120 μm).

As described above, according to the embodiment, since areas of the elements 51-53 are made equal to one another and the electric impedances are made equal to one another, there is an advantage that the transmission sensitivity and reception sensitivity of elements can be made substantially the same. In addition, the arrangement of elements in the elevation direction is MIAE arrangement and the characteristic of the ultrasonic beam can be further improved. Thereby, the image quality and contrast in the near field can be significantly improved.

Further, according to the embodiment, since the interconnections 54 are independently connected to the elements 51-53, an ultrasonic beam with a focal point formed at a desired length can be deflected and transmitted in a desired direction. Thereby, the images can be acquired without change in the position and orientation of the ultrasonic probe, and three-dimensional images can be constructed at a high speed.

Furthermore, according to the embodiment, since the element intervals in the azimuth direction can be taken wider especially in the L1-row, C-row, and R1-row, the crosstalk of ultrasonic waves can be reduced. Therefore, the contrast can be improved in ultrasonic images generated based on the reception signals outputted from the elements.

The method of setting the drive elements to be used when ultrasonic waves are transmitted is the same as that explained in the first embodiment.

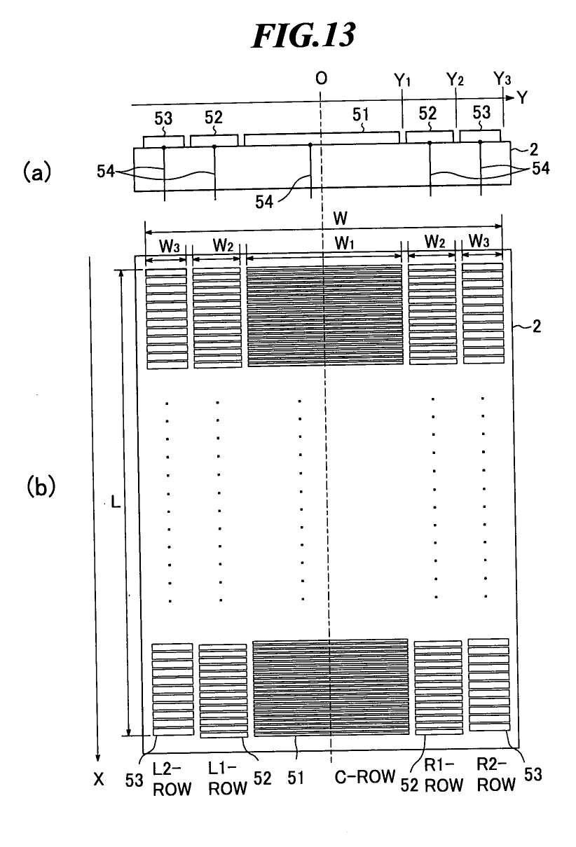

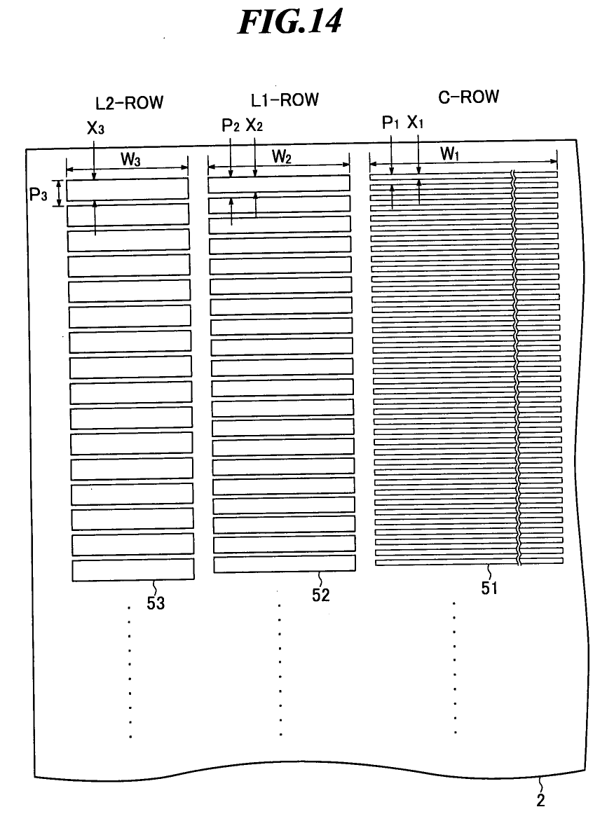

Next, an ultrasonic transducer array according to the sixth embodiment of the present invention will be explained with reference to FIGS. 13 and 14. FIG. 13(a) is a side view showing a state in which the ultrasonic transducer array according to the embodiment is provided on a backing layer, FIG. 13(b) is a plan view thereof, and FIG. 14 is a partially enlarged plan view showing ultrasonic transducers shown in FIG. 13(b).

In the ultrasonic transducer array according to the embodiment, the arrangement of elements is changed from that of the ultrasonic transducer array shown in FIG. 11. That is, the element intervals in the azimuth direction are unified to 30 μm in the C-row where the elements 51 are arranged, in the L1-row and the R1-row where the elements 52 are arranged, and in the L2-row and the R2-row where the elements 53 are arranged. The element interval of 30 μm is designed such that the arrangement pitches P₁-P₃ of the elements containing the lengths X₁ to X₃ of the elements 51-53 are equal to or less than the half of the wavelength of the transmission ultrasonic waves (e.g., 150 μm or less) at the maximum.

The sizes of the respective elements 51-53, the arrangement in the elevation direction (MIAE arrangement), and the interconnection method (independent interconnection) are the same as those in the fifth embodiment. Further, the entire width W of the ultrasonic transducer array is, for example, 5 mm and the length L is, for example, 5 mm.

Specifically, with reference to FIG. 14, 316 channels of elements 51 are arranged at the arrangement pitch P₁=60.7 μm (element length 30.7 μm+element interval 30 μm) in the central C-row. Further, 149 channels of elements 52 are arranged at the arrangement pitch P₂=128.9 μm (element length 98.9 μm+element interval 30 μm) in the L1-row and R1-row. Furthermore, 128 channels of elements 53 are arranged at the arrangement pitch P₃=150 μm (element length 120 μm+element interval 30 μm) in the L2-row and R2-row.

As described above, according to the embodiment, in addition to the improvement in the characteristic of the ultrasonic beam by the MIAE arrangement of elements, the density of elements is made higher toward the center from outside in the elevation direction, and thus, the filling ratio of elements to the entire area of the ultrasonic transducer array can be improved. Thereby, the transmission and reception sensitivity of ultrasonic waves and resolving power can be further improved, and therefore, image quality of ultrasonic images generated in the ultrasonic diagnostic apparatus can be improved.

The method of setting the drive elements to be used when ultrasonic waves are transmitted is the same as that explained in the second embodiment.

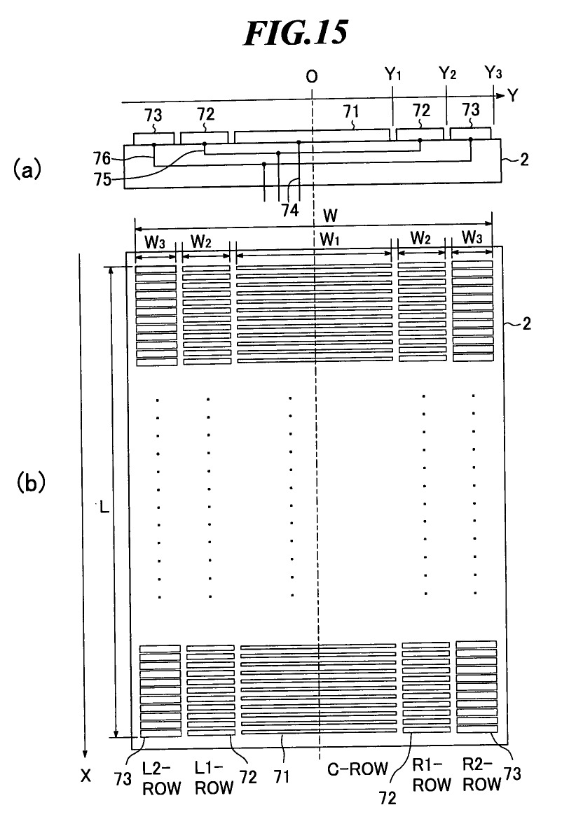

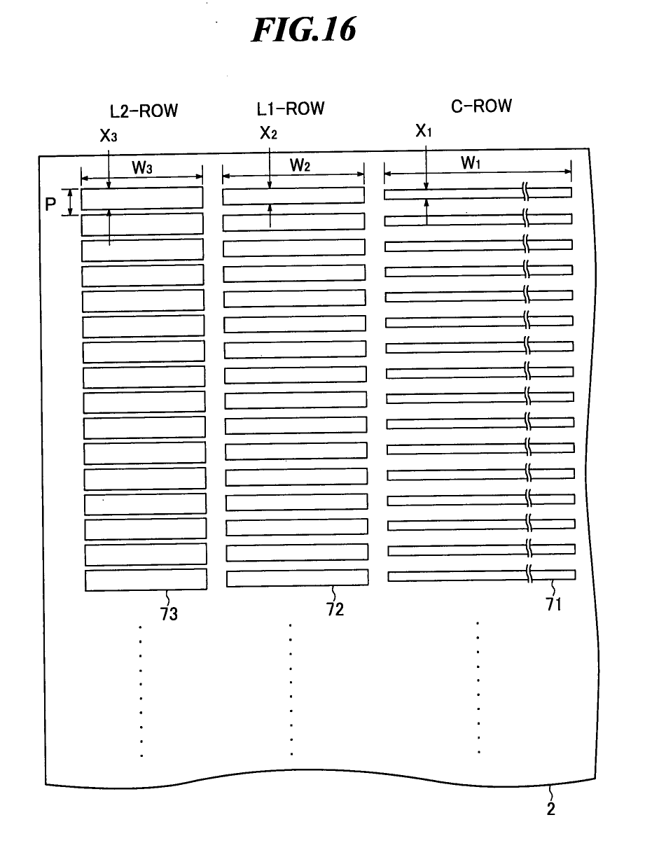

Next, an ultrasonic transducer array according to the seventh embodiment of the present invention will be explained with reference to FIGS. 15 and 16. FIG. 15(a) is a side view showing a state in which the ultrasonic transducer array according to the embodiment is provided on a backing layer, FIG. 15(b) is a plan view thereof, and FIG. 16 is a partially enlarged plan view showing ultrasonic transducers shown in FIG. 15(b).

In the ultrasonic transducer array according to the embodiment, the interconnection method and element size are changed from those of the ultrasonic transducer array shown in FIG. 13.

As shown in FIG. 15(b), the ultrasonic transducer array includes three kinds of elements 71-73 arranged in five rows of L2-row, L1-row, C-row, R1-row, and R2-row. These elements 71-73 are arranged at intervals of 30 μm between the elements arranged in adjacent rows and themselves. Further, the entire width W of the ultrasonic transducer array is 5 mm and the length L is 19 mm.

In each row, 128 channels of elements 71-73 are arranged. The arrangement pitch P of these elements is 150 μm that is equal to or less than the half of the wavelength of the transmission ultrasonic waves.

Further, as shown in FIG. 15(a), the elements 71 in the C-row are connected to interconnections 74, the elements 72 in the L1-row and R1-row are connected to interconnections 75, and the elements 73 in the L2-row and R2-row are connected to interconnections 76. Thus, the elements arranged symmetrically with respect to the center in the elevation direction are commonly interconnected, and thereby, drive signals generated in the ultrasonic diagnostic apparatus main body are supplied to those elements with the same timing.

With reference to FIGS. 15 and 16, the widths W₁ to W₃ and positions Y₁ to Y₃ of the elements 71-73 are designed in the MIAE arrangement. Further, the lengths X₁ to X₃ of the elements 71-73 are designed such that the sums of areas of ultrasonic transmission/reception faces with respect to each interconnection are generally equal to one another according to the widths W₁ to W₃ of the elements 71-73.

Specifically, regarding the size of the element 71 arranged in the C-row, the width W₁ is 2290 μm and the length X₁ is 61.3 μm. Therefore, the area S₁ of the element 71 is obtained by S₁=W₁×X₁=140377≈140400 (μm²). The element interval in the C-row is 88.7 μm (arrangement pitch 150 μm−element length 61.3 μm).

Further, regarding each size of the two elements 72 arranged in the L1-row and R1-row and commonly interconnected, the width W₂is 710 μm and the length X₂ is 98.9 μm. Therefore, the sum of areas S_{2(L+R)} of the two elements 72 is obtained by S_{2(L+R)}=W₂×X₂×2=140438≈140400 (μm²). The element interval in the L1-row and R1-row is 51.1 μm (arrangement pitch 150 μm−element length 98.9 μm).

Furthermore, regarding each size of the two elements 73 arranged in the L2-row and R2-row and commonly interconnected, the width W₃ is 585 μm and the length X₃ is 120 μm. Therefore, the sum of areas S_{3(L+R)} of the two elements 73 is obtained by S_{3(L+R)}=W₃×X₃×2=140400 (μm²). The element interval in the L2-row and R2-row is 30 μm (arrangement pitch 150 μm−element length 120 μm).

As described above, according to the embodiment, since areas of the respective elements are designed such that the total areas of the commonly interconnected elements (one element for the C-row) are generally equal to one another, the synthesized electric impedances with respect to each interconnection are nearly equal to one another, and the characteristics thereof are made substantially the same. Thereby, characteristics such as transmission sensitivity and reception sensitivity of ultrasonic waves can be made equal to one another, and image quality of the ultrasonic images generated in the ultrasonic diagnostic apparatus main body can be improved. Further, the number of interconnections can be reduced in the ultrasonic transducer array with elements in MIAE arrangement by using common interconnections, and therefore, the downsizing of the ultrasonic probe, the thinning down of the cable, and the reduction in costs can be realized.

The method of setting the drive elements to be used when ultrasonic waves are transmitted is the same as that explained in the first embodiment. Further, in the embodiment, the focal length of the ultrasonic beam transmitted in a substantially perpendicular direction to the ultrasonic transmission/reception face can be changed by adjustment of drive timing among the one set of elements that have been set as drive elements. Therefore, ultrasonic images with higher resolving power can be generated by dynamic focus than in the case of using a 1D array.

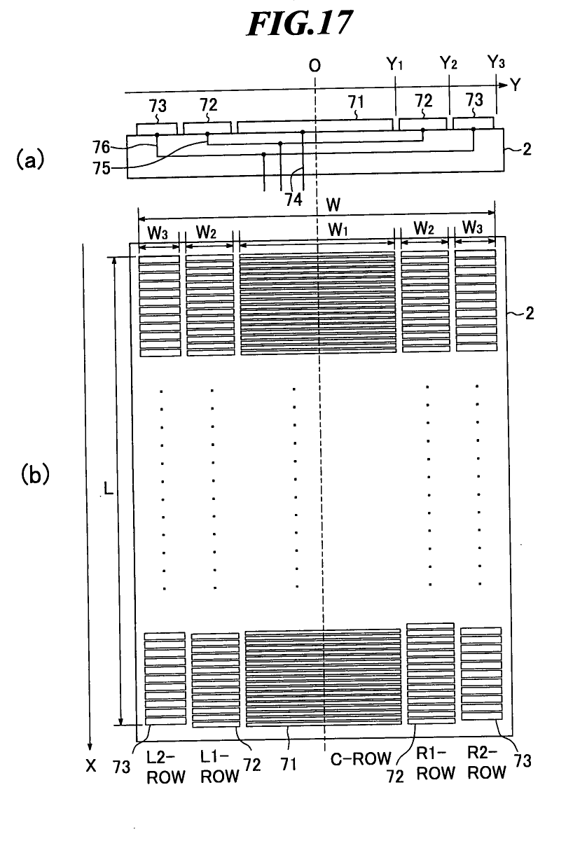

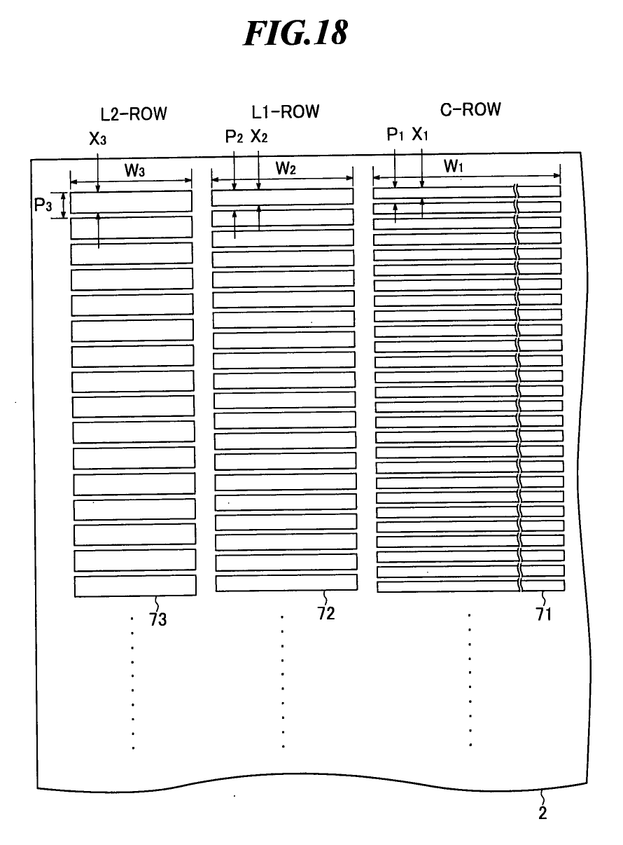

Next, an ultrasonic transducer array according to the eighth embodiment of the present invention will be explained with reference to FIGS. 17 and 18. FIG. 17(a) is a side view showing a state in which the ultrasonic transducer array according to the embodiment is provided on a backing layer, FIG. 17(b) is a plan view thereof, and FIG. 18 is a partially enlarged plan view showing ultrasonic transducers shown in FIG. 17(b).

In the ultrasonic transducer array according to the embodiment, the arrangement of elements is changed from that of the ultrasonic transducer array shown in FIG. 15. That is, the element intervals in the azimuth direction are unified to 30 μm in the C-row where the elements 71 are arranged, in the L1-row and the R1-row where the elements 72 are arranged, and in the L2-row and the R2-row where the elements 73 are arranged. The element interval of 30 μm is designed such that the arrangement pitches P₁-P₃ of the elements containing the lengths X₁ to X₃ of the elements 71-73 are equal to or less than the half of the wavelength of the transmission ultrasonic waves (e.g., 150 μm or less) at the maximum.

The sizes of the respective elements 71-73, the arrangement in the elevation direction (MIAE arrangement), and the interconnection method (common interconnection) are the same as those in the seventh embodiment. Further, the entire width W of the ultrasonic transducer array is, for example, 5 mm and the length L is, for example, 5 mm.

Specifically, with reference to FIG. 18, 210 channels of elements 71 are arranged at the arrangement pitch P₁=91.3 μm (element length 61.3 μm+element interval 30 μm) in the central C-row. Further, 149 channels of elements 72 are arranged at the arrangement pitch P₂=120.5 μm (element length 90.5 μm+element interval 30 μm) in the L1-row and R1-row. Furthermore, 128 channels of elements 73 are arranged at the arrangement pitch P₃=150 μm (element length 120 μm+element interval 30 μm) in the L2-row and R2-row.

As described above, according to the embodiment, in addition to the improvement in the characteristic of the ultrasonic beam by the MIAE arrangement of elements, the density of elements is made higher toward the center from outside in the elevation direction, and thus, the filling ratio of elements to the entire area of the ultrasonic transducer array can be improved. Thereby, the transmission and reception sensitivity of ultrasonic waves and resolving power can be improved, and therefore, image quality of ultrasonic images generated in the ultrasonic diagnostic apparatus main body can be improved. Further, the number of interconnections can be reduced by using common interconnection, and therefore, the downsizing of the ultrasonic probe, the thinning down of the cable, and the reduction in costs can be realized.

The method of setting the drive elements to be used when ultrasonic waves are transmitted is the same as that explained in the second embodiment.

As described above, according to the first to eighth embodiments of the present invention, since the sizes (widths and lengths) of plural kinds of elements having different shapes are defined such that areas thereof are equal to one another, the values of electric impedances of the elements can be made nearly equal to one another. Thereby, the matching characteristics of electric impedances between the ultrasonic diagnostic apparatus main body and themselves can be made substantially the same among the plural kinds of elements, and thus, the characteristics of transmission sensitivity and reception sensitivity of the elements can be made equal to one another. Therefore, ultrasonic waves with fewer variations in transmission intensity for each element (each row) can be transmitted based on the drive signals generated in the ultrasonic diagnostic apparatus main body (see FIG. 3). As a result, ultrasonic beams with good characteristics can be formed and propagated to the object. Further, reception signals with fewer variations in reception sensitivity are inputted to the ultrasonic diagnostic apparatus main body, and thus, gain adjustment for each element (each row) is no longer required in the reception signal processing unit (see FIG. 3). Thereby, processing operation of reception signals can be made simpler.

Therefore, by using an ultrasonic probe or ultrasonic endoscope provided with such an ultrasonic transducer array, good quality ultrasonic images can be easily generated in the ultrasonic diagnostic apparatus main body.

Further, according to the first to eighth embodiments of the present invention, the same piezoelectric material is used for all elements, and the electric impedances are made equal to one another with appropriate element patterns. Therefore, the ultrasonic transducer array according to the embodiments can be easily manufactured by using known technologies including deposition technologies such as screen printing.

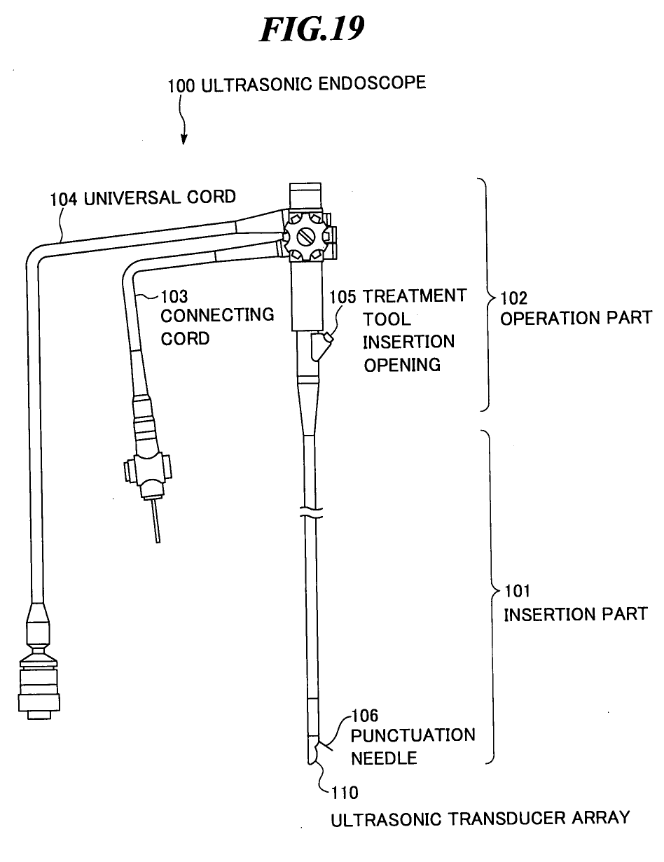

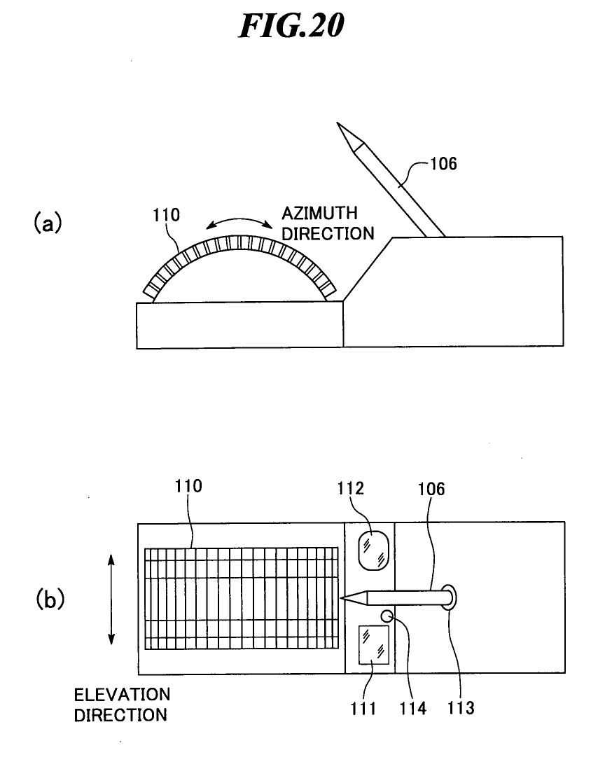

Next, an ultrasonic endoscope according to one embodiment of the present invention will be explained with reference to FIGS. 19 and 20. The ultrasonic transducer array according to the above-mentioned first to eighth embodiments is applicable not only to an ultrasonic probe to be used in contact with the object (see FIG. 2) but also an endoscope to be inserted into the object for use.

FIG. 19 is a schematic diagram showing an appearance of the ultrasonic endoscope. As shown in FIG. 19, the ultrasonic endoscope 100 includes an insertion part 101, an operation part 102, a connecting cord 103, and a universal cord 104.

The insertion part 101 of the ultrasonic endoscope 100 is an elongated tube including a material having flexibility for insertion into the body of the object. The operation part 102 is provided at the base end of the insertion part 101, connected to the ultrasonic diagnostic apparatus main body via the connecting cord 103, and connected to a light source unit via the universal cord 104.

FIG. 20 is an enlarged schematic diagram showing the leading end of the insertion part 101 shown in FIG. 19. FIG. 20(a) is a side view showing the leading end of the insertion part 101, and FIG. 20(b) is a plan view showing the leading end of the insertion part 101.

As shown in FIG. 20, at the leading end of the insertion part 101, an ultrasonic transducer array 110, an observation window 111, an illumination window 112, a treatment tool passage opening 113, and a nozzle hole 114 are provided. Further, a punctuation needle 106 is provided in the treatment tool passage opening 113.

The ultrasonic transducer array 110 is a convex-type multirow array and includes five rows of elements arranged on a curved surface. Any arrangement method of elements (Fresnel arrangement, MIAE arrangement, and so on), shape and size of elements, and interconnection method for the ultrasonic transducer array explained in the first to eighth embodiments may be applied.

Further, as shown in FIG. 20(b), it is desirable that the ultrasonic transducer array 110 is provided such that the elevation direction is perpendicular to the insertion direction of the treatment tool (e.g., the punctuation needle 106) provided in the treatment tool passage opening 113 as seen from above. Thereby, the position of the leading end of the treatment tool in the elevation direction can be detected.

An acoustic matching layer is provided on the ultrasonic transmission face of the ultrasonic transducer array 110, and a backing layer is provided on the opposite face to the ultrasonic transmission face of the ultrasonic transducer array 110. Furthermore, an acoustic lens may be provided on the upper layer of the acoustic matching layer according to need.

An objective lens is fit in the observation window 111, and an input end of an image guide or a solid-state image sensor such as a CCD camera is provided in the imaging position of the objective lens. These configure an observation optical system. Further, an illumination lens for outputting illumination light to be supplied from the light source unit via a light guide is fit in the illumination window 112. These configure an illumination optical system.

The treatment tool passage opening 113 is a hole for leading out a treatment tool inserted from a treatment tool insertion opening 105 (FIG. 19) provided in the operation part 102. Various treatments are performed within a living cavity of the object by projecting the treatment tool such as the punctuation needle 106 or forceps from the hole and operating it in the operation part 102. Furthermore, the nozzle hole 114 is provided for injecting a liquid (water or the like) for cleaning the observation window 111 and the illumination window 112.

In the case where the ultrasonic transducer array explained in the sixth embodiment of the present invention is applied to the ultrasonic endoscope, for example, three-dimensional images on a region of interest with good image quality can be acquired in real time. Referring to such ultrasonic images, a practitioner (a doctor or the like) can accurately grasp the relative position between the treatment tool (e.g., the punctuation needle 106) and an affected part. Thereby, even when the punctuation needle 106 is bent or the insertion direction is deflected from the proper direction, for example, the practitioner can perform treatment accurately and easily.

Here, in FIG. 20, the convex-type multirow array is shown as the ultrasonic transducer array 110, however, a cylindrical (radial-type) multirow array formed by further curving the multirow array in the azimuth direction or a spherical array formed by curving the multirow array not only in the azimuth direction but also in the elevation direction may be applied to the ultrasonic endoscope.

In the above explained embodiments of the present invention, the Fresnel arrangement and MIAE arrangement are used as the arrangements of elements in the elevation direction of the multirow array, however, other arrangements may be used. Further, the number of rows (the number of elements arranged in the elevation direction), the number of columns (the number of elements arranged in the azimuth direction), and the width and length of the entire ultrasonic transducer array may be arbitrarily designed. In this regard, areas of plural kinds of elements having different shapes from one another are made equal to one another, and therefore, the values of impedance characteristics of the elements can be made nearly equal to one another, and the electric impedances can be made substantially the same.

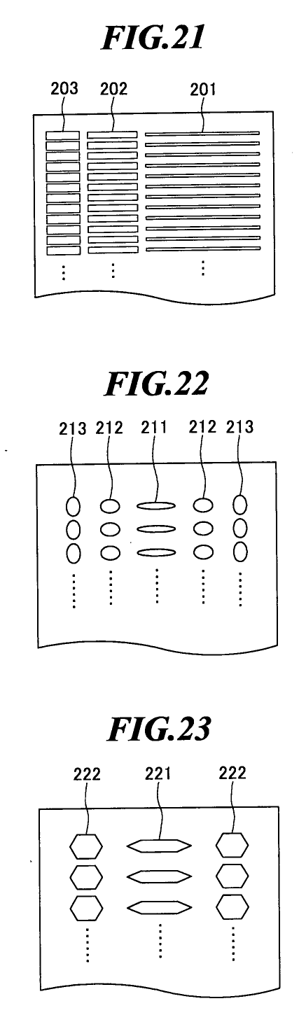

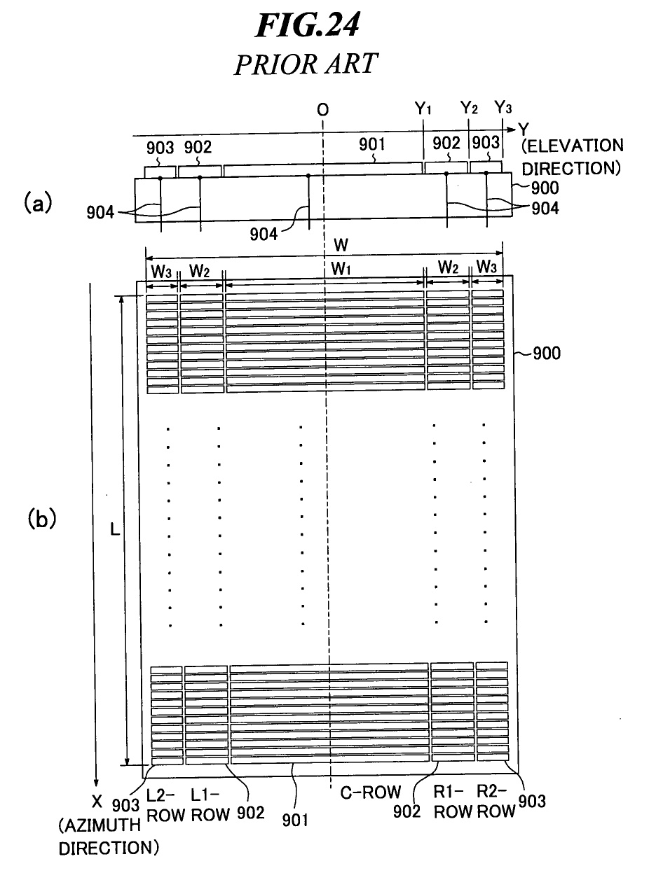

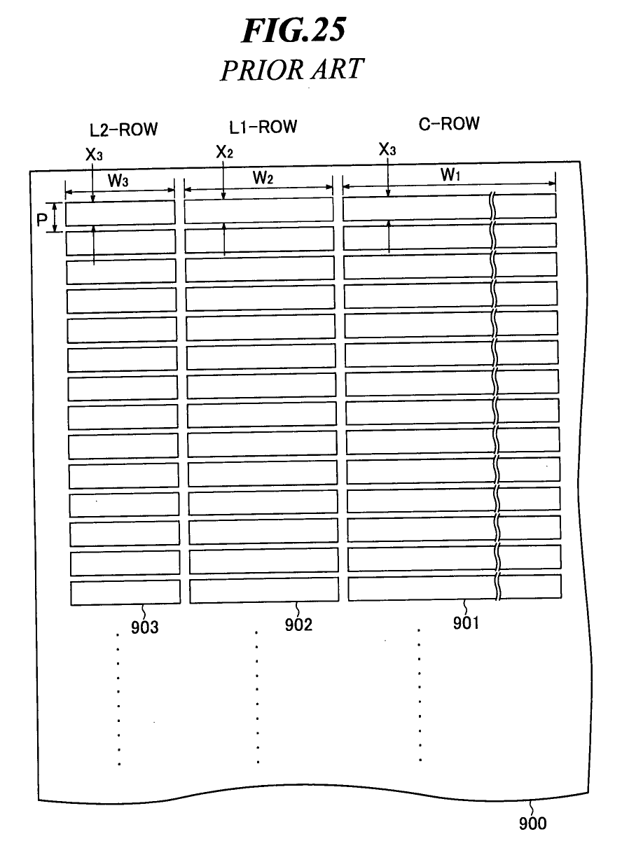

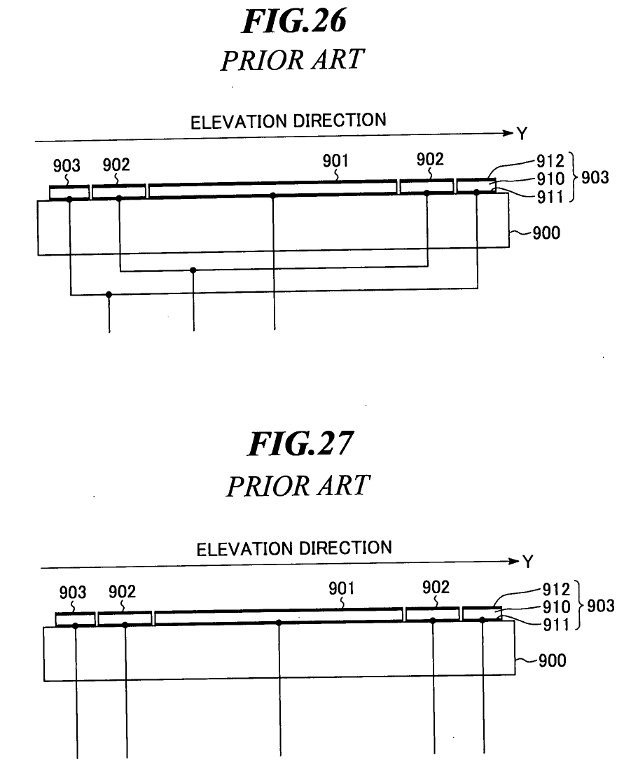

Here, in the above-mentioned embodiments, the multirow array, in which the arrangement is symmetric with respect to the center of the elevation direction, is explained. However, the elements are not necessarily arranged symmetrically. For example, as shown in FIG. 21, the present invention may be applied to an ultrasonic transducer array in which plural kinds of elements 201-203 are asymmetrically arranged.

Further, the number of rows, in which the elements are arranged, is not limited to five, but the present invention may be applied to a multirow array having at least two rows. Furthermore, in the above-mentioned embodiments, the arrangement pitch in the elevation direction is equal to or more than the wavelength of transmission ultrasonic waves, however, it may be less than the wavelength of transmission ultrasonic waves.

Further, regarding the shape of elements, not only the rectangular shape but also an arbitrary shape may be used. For example, as shown in FIG. 22, oval elements 211-213 may be arranged in multirow. Alternatively, as shown in FIG. 23, polygonal (e.g., hexagonal) elements 221 and 222 may be arranged in multirow. Alternatively, plural kinds of elements having largely different shapes from one another (e.g., oval elements and polygonal elements) may be arranged in one ultrasonic transducer array. In any case, the elements are designed such that areas of plural kinds of elements having different shapes are made equal to one another, and therefore, the electric impedance characteristics can be made substantially the same among those elements, and thereby, characteristics such as sensitivity can be made equal to one another.

Alternatively, when plural elements are commonly interconnected, the sizes of the plural kinds of elements and interconnections are designed such that the total areas of the commonly interconnected elements are equal to one another, and thereby, the same effect can be obtained. The number of commonly interconnected elements may be larger than two. Further, in the case of interconnection, the shapes are not necessarily different among elements that are not commonly connected. For example, the total areas may be made equal to one another by commonly interconnecting a first element to plural second elements each of which is similar to the first element.

Furthermore, the arrangement surface of elements in the ultrasonic transducer array may be a flat surface as shown in FIG. 2, a convex surface as shown in FIG. 20, a concave surface, a spherical surface, or any other curved surfaces.

1. An ultrasonic transducer array in which plural ultrasonic transducers having at least two kinds of shapes are arranged, said ultrasonic transducer array comprising:
a first ultrasonic transducer; and
a second ultrasonic transducer having a different shape from that of said first ultrasonic transducer, an area of an ultrasonic transmission/reception face of said second ultrasonic transducer being substantially equal to an area of an ultrasonic transmission/reception face of said first ultrasonic transducer.

2. The ultrasonic transducer array according to claim 1, wherein an electric impedance of said first ultrasonic transducer and an electric impedance of said second ultrasonic transducer are substantially equal to each other.

3. An ultrasonic transducer array in which plural ultrasonic transducers are arranged, said ultrasonic transducer array comprising:
at least one first ultrasonic transducer commonly connected to a first interconnection; and
plural second ultrasonic transducers commonly connected to a second interconnection, a sum of areas of ultrasonic transmission/reception faces of said plural second ultrasonic transducers being substantially equal to a sum of areas of ultrasonic transmission/reception faces of said at least one first ultrasonic transducer.

4. The ultrasonic transducer array according to claim 3, wherein a synthesized electric impedance of said at least one first ultrasonic transducer and a synthesized electric impedance of said plural second ultrasonic transducers are substantially equal to each other.

5. The ultrasonic transducer array according to claim 1, comprising:
plural first ultrasonic transducers one-dimensionally arranged in a first element row; and
plural second ultrasonic transducers one-dimensionally arranged in a second element row in parallel with the first element row.

6. The ultrasonic transducer array according to claim 3, comprising:
plural first ultrasonic transducers one-dimensionally arranged in a first element row; and
plural second ultrasonic transducers one-dimensionally arranged in a second element row in parallel with the first element row.

7. The ultrasonic transducer array according to claim 5, wherein each of said plural first and second ultrasonic transducers has a rectangular ultrasonic transmission/reception face.

8. The ultrasonic transducer array according to claim 5, wherein a width of an ultrasonic transducer arranged in an outer element row is narrower than a width of an ultrasonic transducer arranged in an inner element row in an orientation perpendicular to an arrangement direction of ultrasonic transducers in each element row.

9. The ultrasonic transducer array according to claim 5, wherein an arrangement pitch of said plural first ultrasonic transducers in the first element row and an arrangement pitch of said plural second ultrasonic transducers in the second element row are equal to each other.

10. The ultrasonic transducer array according to claim 5, wherein an interval between said plural first ultrasonic transducers in the first element row and an interval between said plural second ultrasonic transducers in the second element row are equal to each other.

11. The ultrasonic transducer array according to claim 5, wherein density of ultrasonic transducers arranged in an inner element row is higher than density of ultrasonic transducers arranged in an outer element row in an orientation perpendicular to an arrangement direction of ultrasonic transducers in each element row.

12. An ultrasonic probe comprising:
an ultrasonic transducer array in which plural ultrasonic transducers having at least two kinds of shapes are arranged, said ultrasonic transducer array including a first ultrasonic transducer, and a second ultrasonic transducer having a different shape from that of said first ultrasonic transducer, an area of an ultrasonic transmission/reception face of said second ultrasonic transducer being substantially equal to an area of an ultrasonic transmission/reception face of said first ultrasonic transducer;
an acoustic matching layer provided at an ultrasonic transmission surface side of said ultrasonic transducer array; and
a backing layer provided at an opposite side to said ultrasonic transmission surface side of said ultrasonic transducer array.

13. An ultrasonic probe comprising:
an ultrasonic transducer array in which plural ultrasonic transducers are arranged, said ultrasonic transducer array including at least one first ultrasonic transducer commonly connected to a first interconnection, and plural second ultrasonic transducers commonly connected to a second interconnection, a sum of areas of ultrasonic transmission/reception faces of said plural second ultrasonic transducers being substantially equal to a sum of areas of ultrasonic transmission/reception faces of said at least one first ultrasonic transducer;
an acoustic matching layer provided at an ultrasonic transmission surface side of said ultrasonic transducer array; and
a backing layer provided at an opposite side to said ultrasonic transmission surface side of said ultrasonic transducer array.

14. An ultrasonic endoscope to be inserted into a body of an object to be inspected, said ultrasonic endoscope comprising:
an insertion part including a material having flexibility and to be inserted into the body of the object; and
an ultrasonic transducer array, in which plural ultrasonic transducers having at least two kinds of shapes are arranged, provided at a leading end of said insertion part, said ultrasonic transducer array including a first ultrasonic transducer, and a second ultrasonic transducer having a different shape from that of said first ultrasonic transducer, an area of an ultrasonic transmission/reception face of said second ultrasonic transducer being substantially equal to an area of an ultrasonic transmission/reception face of said first ultrasonic transducer.

15. An ultrasonic endoscope to be inserted into a body of an object to be inspected, said ultrasonic endoscope comprising:
an insertion part including a material having flexibility and to be inserted into the body of the object; and
an ultrasonic transducer array, in which plural ultrasonic transducers are arranged, provided at a leading end of said insertion part, said ultrasonic transducer array including at least one first ultrasonic transducer commonly connected to a first interconnection, and plural second ultrasonic transducers commonly connected to a second interconnection, a sum of areas of ultrasonic transmission/reception faces of said plural second ultrasonic transducers being substantially equal to a sum of areas of ultrasonic transmission/reception faces of said at least one first ultrasonic transducer.

16. The ultrasonic endoscope according to claim 14, further comprising:
a treatment tool to be inserted from an opening provided at the leading end of said insertion part through an interior of said insertion part into the body of the object;
wherein said ultrasonic transducer array includes plural first ultrasonic transducers one-dimensionally arranged in a first element row, and plural second ultrasonic transducers one-dimensionally arranged in a second element row in parallel with the first element row, and plural ultrasonic transducers in each element row are arranged so as to detect a position of a leading end of said treatment tool.

17. The ultrasonic endoscope according to claim 15, further comprising:
a treatment tool to be inserted from an opening provided at the leading end of said insertion part through an interior of said insertion part into the body of the object;
wherein said ultrasonic transducer array includes plural first ultrasonic transducers one-dimensionally arranged in a first element row, and plural second ultrasonic transducers one-dimensionally arranged in a second element row in parallel with the first element row, and plural ultrasonic transducers in each element row are arranged so as to detect a position of a leading end of said treatment tool.

18. An ultrasonic diagnostic apparatus comprising:
an ultrasonic probe including an ultrasonic transducer array in which plural ultrasonic transducers having at least two kinds of shapes are arranged, said ultrasonic transducer array including a first ultrasonic transducer, and a second ultrasonic transducer having a different shape from that of said first ultrasonic transducer, an area of an ultrasonic transmission/reception face of said second ultrasonic transducer being substantially equal to an area of an ultrasonic transmission/reception face of said first ultrasonic transducer, an acoustic matching layer provided at an ultrasonic transmission surface side of said ultrasonic transducer array, and a backing layer provided at an opposite side to said ultrasonic transmission surface side of said ultrasonic transducer array;
a drive signal generating unit for generating drive signals to be supplied to at least said first and second ultrasonic transducers;
a signal processing unit for processing reception signals outputted from at least said first and second ultrasonic transducers; and
an image generating unit for generating an ultrasonic image based on the reception signals processed by said signal processing unit.

19. An ultrasonic diagnostic apparatus comprising:
an ultrasonic probe including an ultrasonic transducer array in which plural ultrasonic transducers are arranged, said ultrasonic transducer array including at least one first ultrasonic transducer commonly connected to a first interconnection, and plural second ultrasonic transducers commonly connected to a second interconnection, a sum of areas of ultrasonic transmission/reception faces of said plural second ultrasonic transducers being substantially equal to a sum of areas of ultrasonic transmission/reception faces of said at least one first ultrasonic transducer, an acoustic matching layer provided at an ultrasonic transmission surface side of said ultrasonic transducer array, and a backing layer provided at an opposite side to said ultrasonic transmission surface side of said ultrasonic transducer array;
a drive signal generating unit for generating drive signals to be supplied to at least said first and second ultrasonic transducers;
a signal processing unit for processing reception signals outputted from at least said first and second ultrasonic transducers; and
an image generating unit for generating an ultrasonic image based on the reception signals processed by said signal processing unit.

20. An ultrasonic diagnostic apparatus comprising:
an ultrasonic endoscope to be inserted into a body of an object to be inspected, said ultrasonic endoscope including an insertion part including a material having flexibility and to be inserted into the body of the object, and an ultrasonic transducer array, in which plural ultrasonic transducers having at least two kinds of shapes are arranged, provided at a leading end of said insertion part, said ultrasonic transducer array including a first ultrasonic transducer, and a second ultrasonic transducer having a different shape from that of said first ultrasonic transducer, an area of an ultrasonic transmission/reception face of said second ultrasonic transducer being substantially equal to an area of an ultrasonic transmission/reception face of said first ultrasonic transducer;
a drive signal generating unit for generating drive signals to be supplied to at least said first and second ultrasonic transducers;
a signal processing unit for processing reception signals outputted from at least said first and second ultrasonic transducers; and
an image generating unit for generating an ultrasonic image based on the reception signals processed by said signal processing unit.

21. An ultrasonic diagnostic apparatus comprising:
an ultrasonic endoscope to be inserted into a body of an object to be inspected, said ultrasonic endoscope including an insertion part including a material having flexibility and to be inserted into the body of the object; and an ultrasonic transducer array, in which plural ultrasonic transducers are arranged, provided at a leading end of said insertion part, said ultrasonic transducer array including at least one first ultrasonic transducer commonly connected to a first interconnection, and plural second ultrasonic transducers commonly connected to a second interconnection, a sum of areas of ultrasonic transmission/reception faces of said plural second ultrasonic transducers being substantially equal to a sum of areas of ultrasonic transmission/reception faces of said at least one first ultrasonic transducer;
a drive signal generating unit for generating drive signals to be supplied to at least said first and second ultrasonic transducers;
a signal processing unit for processing reception signals outputted from at least said first and second ultrasonic transducers; and
an image generating unit for generating an ultrasonic image based on the reception signals processed by said signal processing unit.
